SEQUENCE LISTING (42 sequences)

SEQ ID NO: 1 - protein, 137 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAK

SEQ ID NO: 2 - protein, 137 aa, Adeno-associated virus
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVK

SEQ ID NO: 3 - protein, 137 aa, Adeno-associated virus
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAK

SEQ ID NO: 4 - protein, 136 aa, Adeno-associated virus
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGE

SEQ ID NO: 5 - protein, 136 aa, Adeno-associated virus
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAK

SEQ ID NO: 6 - protein, 137 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAK

SEQ ID NO: 7 - protein, 137 aa, Adeno-associated virus
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAK

SEQ ID NO: 8 - protein, 137 aa, Adeno-associated virus
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAK

SEQ ID NO: 9 - protein, 137 aa, Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAK

SEQ ID NO: 10 - protein, 137 aa, Adeno-associated virus; identical to SEQ ID NO: 1

SEQ ID NO: 11 - protein, 137 aa, Adeno-associated virus; identical to SEQ ID NO: 1

SEQ ID NO: 12 - protein, 137 aa, Adeno-associated virus
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVK

SEQ ID NO: 13 - protein, 136 aa, Adeno-associated virus
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQERLQEDTSFGGNLGRAVFQAKKRILEPLGLVEEAAK

SEQ ID NO: 14 - protein, 736 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 15 - protein, 735 aa, Adeno-associated virus [sequence omitted]

SEQ ID NO: 16 - protein, 736 aa, Adeno-associated virus
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 17 - protein, 734 aa, Adeno-associated virus
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 18 - protein, 724 aa, Adeno-associated virus
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 19 - protein, 736 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 20 - protein, 737 aa, Adeno-associated virus
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 21 - protein, 738 aa, Adeno-associated virus
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 22 - protein, 736 aa, Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 23 - protein, 738 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 24 - protein, 733 aa, Adeno-associated virus
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 25 - protein, 742 aa, Adeno-associated virus
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEMSHDAEMRAAPGGNAVEAGQGADGVGNASGDWHCDSTWSEGRVTTTSTRTWVLPTYNNHLYLRIGTTANSNTYNGFSTPWGYFDFNRFHCHFSPRDWQRLINNNWGLRPKSMRVKIFNIQVKEVTTSNGETTVANNLTSTVQIFADSTYELPYVMDAGQEGSFPPFPNDVFMVPQYGYCGVVTGKNQNQTDRNAFYCLEYFPSQMLRTGNNFEVSYQFEKVPFHSMYAHSQSLDRMMNPLLDQYLWHLQSTTTGNSLNQGTATTTYGKITTGDFAYYRKNWLPGACIKQQKFSKNANQNYKIPASGGDALLKYDTHTTLNGRWSNMAPGPPMATAGAGDSDFSNSQLIFAGPNPSGNTTTSSNNLLFTSEEEIATTNPRDTDMFGQIADNNQNATTAPHIANLDAMGIVPGMVWQNRDIYYQGPIWAKVPHTDGHFHPSPLMGGFGLKHPPPQIFIKNTPVPANPNTTFSAARINSFLTQYSTGQVAVQIDWEIQKEHSKRWNPEVQFTSNYGTQNSMLWAPDNAGNYHELRAIGSRFLTHHL

SEQ ID NO: 26 - protein, 733 aa, Adeno-associated virus
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQERLQEDTSFGGNLGRAVFQAKKRILEPLGLVEEAAKTAPGKKRPVEQSPAEPDSSSGIGKSGQQPARKRLNFGQTGDTESVPDPQPLGQPPAAPSGVGSTTMASGGGAPMADNNEGADGVGNSSGNWHCDSQWLGDRVITTSTRTWALPTYNNHLYKQISSQSGATNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYHLNGRDSLVNPGPAMASHKDDKEKFFPMHGTLIFGKEGTNANNADLENVMITDEEEIRTTNPVATEQYGTVSNNLQNSNAGPTTGTVNHQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 27 - protein, 725 aa, Artificial Sequence
  Synthetic Polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 28 - protein, 725 aa, Artificial Sequence
  Synthetic Polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 29 - protein, 736 aa, Artificial Sequence
  Synthetic Polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 30 - protein, 736 aa, Artificial Sequence
  Synthetic Polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 31 - protein, 5 aa, Artificial Sequence
  Synthetic Polypeptide
  misc_feature (3)..(4): Xaa can be any naturally occurring amino acid
HDXXY

SEQ ID NO: 32 - protein, 5 aa, Artificial Sequence
  Synthetic Polypeptide
HDKAY

SEQ ID NO: 33 - protein, 5 aa, Artificial Sequence
  Synthetic Polypeptide
HDISY

SEQ ID NO: 34 - protein, 5 aa, Artificial Sequence
  Synthetic Polypeptide
  misc_feature (2)..(2): Xaa can be any naturally occurring amino acid
  misc_feature (4)..(4): Xaa can be any naturally occurring amino acid
YXGXG

SEQ ID NO: 35 - protein, 9 aa, Artificial Sequence
  Synthetic Polypeptide
KRVLEPLGL

SEQ ID NO: 36 - protein, 12 aa, Artificial Sequence
  Synthetic Polypeptide
LNFGQTGDADSV

SEQ ID NO: 37 - DNA, 2184 bp, Artificial Sequence
  Synthetic Polynucleotide
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaatctaga

SEQ ID NO: 38 - DNA, 2178 bp, Artificial Sequence
  Synthetic Polynucleotide
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 39 - DNA, 2217 bp, Artificial Sequence
  Synthetic Polynucleotide
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaatctaga

SEQ ID NO: 40 - DNA, 2217 bp, Artificial Sequence
  Synthetic Polynucleotide
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaatctaga

SEQ ID NO: 41 - protein, 738 aa, Artificial Sequence
  Synthetic Polypeptide
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 42 - DNA, 2223 bp, Artificial Sequence
  Synthetic Polynucleotide
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaatctaga

(19) United States
(12) Patent Application Publication
(10) Pub. No.: US 2021/0292373 A1
(43) Pub. Date: Sep. 23, 2021

(54) AAV VP1U CHIMERAS

(71) Applicant: University of Florida Research Foundation, Incorporated, Gainesville, FL (US)

(72) Inventors: Mavis Agbandje-McKenna, Gainesville, FL (US); Robert McKenna, Gainesville, FL (US); Antonette D. Bennett, Gainesville, FL (US)

(73) Assignee: University of Florida Research Foundation, Incorporated, Gainesville, FL (US)

(21) Appl. No.: 17/258,898

(22) PCT Filed: Jul. 10, 2019
(86) PCT No.: PCT/US2019/041251

§ 371 Date: Jan. 8, 2021

Related U.S. Application Data
(60) Provisional application No. 62/696,320, filed on Jul. 10, 2018.

(51) Int. Cl. C07K 14/015; C12N 7/00

(52) U.S. Cl. CPC C07K 14/015; C12N 2750/14121; C12N 2750/14122; C12N 7/00

(57) ABSTRACT

Disclosed herein are chimeric recombinant adeno-associated virus (rAAV) capsid proteins comprising a region of VP1μ that is replaced by the corresponding amino acids of the VP1μ of AAV serotype 1 (AAV1) or AAV serotype 8 (AAV8), and particles comprising them.

### RELATED APPLICATIONS

This application claims the benefit of U.S. provisional application No. 62/696,320, filed Jul. 10, 2018, the entire contents of which are incorporated by reference herein.

### BACKGROUND

While recombinant adeno-associated virus (rAAV) mediated gene therapy holds much promise for a variety of indications, significant challenges in delivering genes using rAAV particles exist, including difficulties in delivering genes efficiently to certain target cells/tissues and avoiding the detrimental effects of the pre-existing host immunity to AAV capsids.

### SUMMARY

The present disclosure focuses on AAV capsid protein (VP) determinants of specific tissue tropism and transduction (gene expression) efficiency. The present invention relates, at least in part, to the discovery through biophysical studies that AAV VP1 N-terminal unique (VP1u) region has to unfold in order to get exposed and then refold to facilitate trafficking of capsids through the endosomal pathway, and that the VP1u of certain AAV serotypes unfold much faster than others. The present invention also relates to the development of rAAV chimeras having a VP1u region of different AAV serotypes that show enhanced transduction efficiency than their wild-type counterparts.

Accordingly provided herein is a chimeric recombinant adeno-associated virus (rAAV) particle comprising a chimeric VP1 capsid protein comprising a backbone of a first serotype and a region of VP1u of a second serotype. In some embodiments, the second serotype is different from the first serotype. In some embodiments, the second serotype is AAV serotype 1 (AAV1) or AAV serotype 8 (AAV8). In some embodiments, the chimeric particle has a greater transduction efficiency relative to its wild-type counterpart as measured in a cell.

In some embodiments, the first serotype is 1, 2, 5, 8 or 9. In some embodiments, the second serotype is AAV1 or AAV8.

In some embodiments, the region of VP1u of the second serotype is at least 5 amino acids long. In some embodiments, the region of VP1u of the second serotype is at least 50 amino acids long. In some embodiments, the region of VP1u of the second serotype is at least 100 amino acids long.

In some embodiments, the region of VP1u that is of a second serotype is the entire VP1u. In some embodiments, the region of VP1u of the second serotype is amino acids 1-137 of AAV1 or AAV8. In some embodiments, a rAAV particle comprises a region of VP1u of the second serotype that is the VP1u of AAV1 (1VP1u). In some embodiments, and the sequence of 1VP1u is SEQ ID NO: 1. In some embodiments, a rAAV particle comprises a region of VP1u of the second serotype that is the VP1u of AAV8 (8VP1u). In some embodiments, and the sequence of 1VP1u is SEQ ID NO: 8. In some embodiments, the region of VP1u of the second serotype comprises a phospholipase A2 domain.

In some embodiments, the first serotype is serotype 5.

In some embodiments, the region of VP1u of the second serotype comprises residues D24, K84, or both D24 and K84 of AAV1, or the equivalent positions in other AAV serotypes. In some embodiments, the region of VP1u of the second serotype comprises residues A24, Q84, or both A24 and Q84 of AAV8, or the equivalent positions in other AAV serotypes.

In some embodiments, the chimeric particle has a transduction efficiency that is at least 20% greater relative to its wild-type counterpart. In some embodiments, the chimeric particle has a transduction efficiency that is greater by at least 4 times relative to its wild-type counterpart. In some embodiments, the cell is selected from the group consisting of: HEK293, HEPG2, LEC2, PRO5, ARPE19, and COS7.

In some aspects, provided herein are nucleic acid that encode any one of the chimeric VP1 capsid proteins disclosed herein. In some embodiments, such a nucleic acid is comprised by a nucleic acid vector (e.g., a plasmid, or virus vector).

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIGS. 1A and 1B show circular dichroism (CD) spectra for AAV1, AAV2, AAV5, and AAV8 (FIG. 1A) and stability (FIG. 1B) for AAV1, AAV2, AAV5, AAV8, and AAV9 at pHs associated with endosomal trafficking: pH 7.4, 6.0, 5.5, and 4.0.

FIG. 2 shows AAV VP capsid protein sequence alignment. The calcium binding site (YXGXG, SEQ ID NO: 34); PLA2 domain (HDXXY, SEQ ID NO: 31), A1 (123-KRVLEPLGL-131, SEQ ID NO: 35) and A69 (169-LNFGQTGDADSV-184, SEQ ID NO: 36) are epitopes are indicated along with the VP2 and VP3 N-termini. From top to bottom, sequences correspond to SEQ ID NOs: 1, 2, 5, 6, 8, and 9.

FIG. 3 shows sequence alignment of AAV1 to AAV13 VP1u. “*” denotes Positions which have a single, fully conserved residue. “:” denotes conservation between groups of strongly similar properties. “.” denotes conservation between groups of weakly similar properties. From top to bottom, sequences correspond to SEQ ID NOs: 1-13.

FIGS. 4A-4E show negative stain EMs, silver stained gel, as well as transduction phenotypes of the following chimeric rAAV particles relative to their wild type counterparts in various cell lines: (FIG. 4A) AAV1-8VP1u, (FIG. 4B) AAV2-1VP1u and AAV2-8VP1u, (FIG. 4C) AAV5-1VP1u and AAV5-8VP1u, (FIG. 4D) AAV8-1VP1u, and (FIG. 4E) AAV9-1VP1u and AAV9-8VP1u.

### DETAILED DESCRIPTION

The AAV VP1 N-terminal unique (VP1u) is essential for host infection. It contains a phospholipase A2 (PLA2) domain that functions to enable the virus to escape from the endosome/lysosome pathway during cellular trafficking and nuclear localization signals (NLS) for delivery of genome-containing virions to the nucleus for replication. AAV VP1u also plays a role in cell sorting (e.g., moving from one compartment of a cell to another) and signal transduction. It was found that to carry out one or more of these functions, VP1u of different AAV serotypes undergo structural rearrangements at different rates to become externalized from its inside location (i.e., within the capsid) onto the capsid surface, an event that is dependent on cellular pH and cation environment.

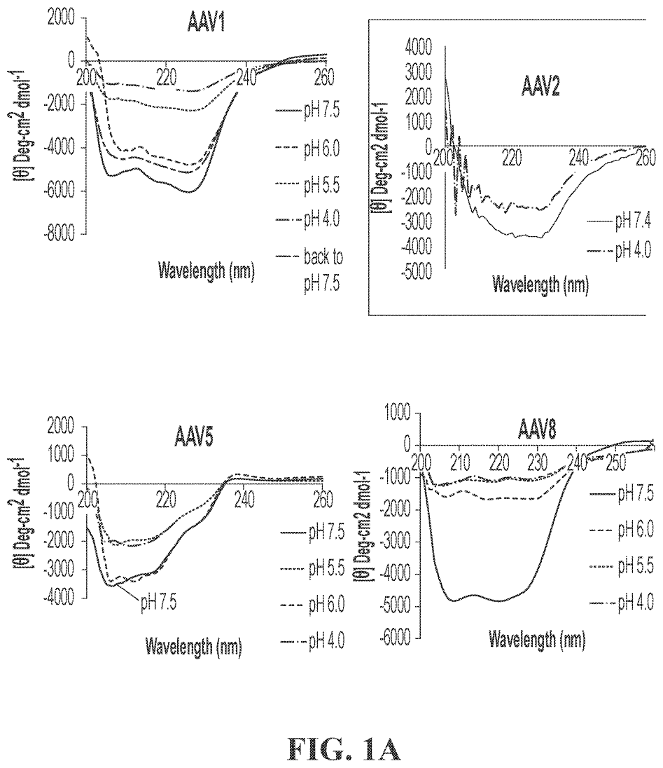

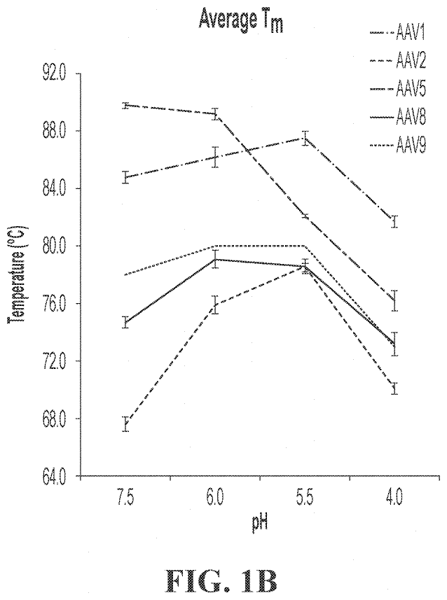

It was observed that, in comparative biochemical and biophysical studies of several AAV serotypes (AAV1/6, AAV2, AAV5, AAV8) selected to span the spectrum of similarity and differences in sequence and structure, there is a difference in (i) pH-dependent capsid stability, (ii) pH-dependent secondary structural transitions of VP1u unfolding (FIG. 1), and (iii) VP1u exposure dynamics. Accordingly, they developed chimeric rAAV particles in which the VP1u, or portion thereof, are replaced with VP1u, or portions thereof, of other serotypes that result in improved cellular transduction efficiency.

Accordingly provided herein are chimeric rAAV capsid proteins comprising a region of VP1u that is replaced by the corresponding amino acids of the VP1u regions of serotypes that result in improved transduction efficiency.

### AAV Structure

The wild-type AAV genome is a single-stranded deoxyribonucleic acid (ssDNA), either positive- or negative-sensed. The genome comprises two inverted terminal repeats (ITRs), one at each end of the DNA strand, and two open reading frames (ORFs): rep and cap between the ITRs. The rep ORF comprises four overlapping genes encoding Rep proteins required for the AAV life cycle: Rep78, Rep68, Rep52 and Rep40. The cap ORF comprises overlapping genes encoding capsid proteins: VP1, VP2 and VP3, which interact together to form the viral capsid. VP1, VP2 and VP3 are translated from one mRNA transcript, which can be spliced in two different manners: either a longer or shorter intron can be excised resulting in the formation of two isoforms of mRNAs: a ˜2.3 kb- and a ˜2.6 kb-long mRNA isoform. The capsid forms a supramolecular assembly of approximately 60 individual capsid protein subunits into a non-enveloped, T-1 icosahedral lattice capable of protecting the AAV genome. The mature capsid is composed of VP1. VP2, and VP3 (molecular masses of approximately 87, 73, and 62 kDa respectively) in a ratio of about 1:1:10.

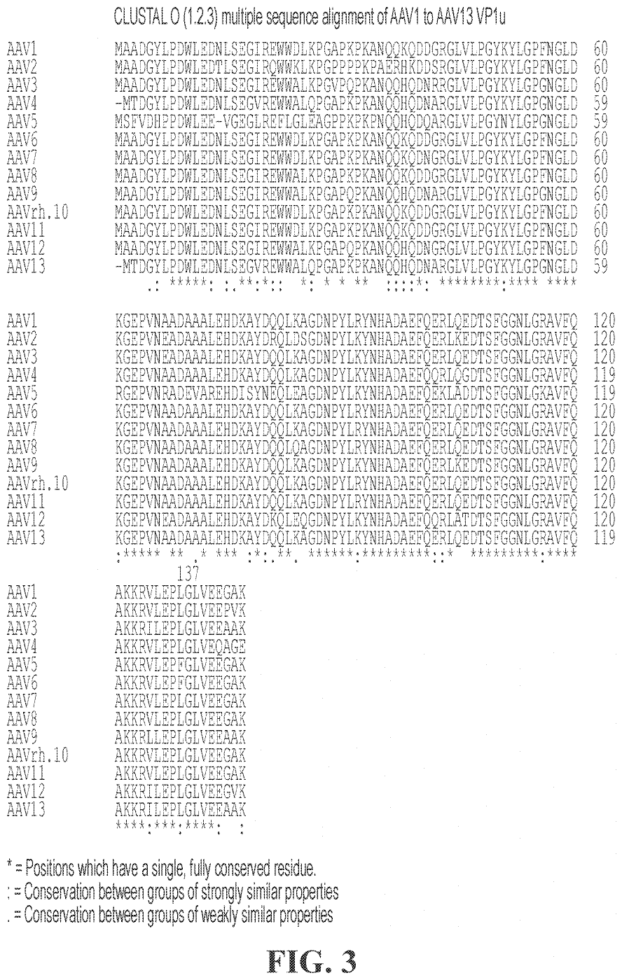

Generally, VP1 has 735 amino acids. AAV5 VP1 has 724 amino acids. For most serotypes (e.g, AAV1, AAV2, AAV8 or AAV9), capsid protein VP2 is made up of amino acids 138-735, 138-736, or 138-738 of VP1. In some serotypes, e.g., AAV5, capsid protein VP2 is made up of amino acids 137-724 of VP1. For most serotypes, capsid protein VP3 is made up of amino acids 203-735, 203-736, or 203-738 of VP1. Thus, VP1 has an N-terminal region that is unique to only VP1. This region is referred to as VP1u region. The VP1u region of most AAV serotypes (e.g., AAV1, AAV2, AAV3, AAV6, AAV7, AAV8, AAV9, AAVrh.10, AAV11, or AAV12) is 137 amino acids long and on the N-terminus end of VP1 capsid protein. For AAV4, AAV5, and AAV13, the VP1u is 136 amino acids long. FIG. 3 shows the alignment between the VP1u regions of AAV serotypes 1-13. SEQ ID NOs: 1-13 provide examples for sequences of the VP1u of AAV serotypes 1, 2, 3, 4, 5, 6, 7, 8, 9, rh.10, 11, 12, and 13.

Example of AAV1 VP1u (1VP1u) capsid protein
sequence
(SEQ ID NO: 1)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAK
Example of AAV2 VP1u (2VP1u) capsid protein
sequence
(SEQ ID NO: 2)
MAADGYLPDWLEDTLSEGIRQWWKLKPGPPPPKPAERHKDDSRGLVLPGY
KYLGPFNGLDKGEPVNEADAAALEHDKAYDRQLDSGDNPYLKYNHADAEF
QERLKEDTSFGGNLGRAVFQAKKRVLEPLGLVEEPVK
Example of AAV3 VP1u (3VP1u) capsid protein
sequence
(SEQ ID NO: 3)
MAADGYLPDWLEDNLSEGIREWWALKPGVPQPKANQQHQDNRRGLVLPGY
KYLGPGNGLDKGEPVNEADAAALEHDKAYDQQLKAGDNPYLKYNHADAEF
QERLQEDTSFGGNLGRAVFQAKKRILEPLGLVEEAAK
Example of AAV4 VP1u (4VP1u) capsid protein
sequence
(SEQ ID NO: 4)
MTDGYLPDWLEDNLSEGVREWWALQPGAPKPKANQQHQDNARGLVLPGYK
YLGPGNGLDKGEPVNAADAAALEHDKAYDQQLKAGDNPYLKYNHADAEFQ
QRLQGDTSFGGNLGRAVFQAKKRVLEPLGLVEQAGE
Example of AAV5 VP1u (5VP1u) capsid protein
sequence
(SEQ ID NO: 5)
MSFVDHPPDWLEEVGEGLREFLGLEAGPPKPKPNQQHQDQARGLVLPGYN
YLGPGNGLDRGEPVNRADEVAREHDISYNEQLEAGDNPYLKYNHADAEFQ
EKLADDTSFGGNLGKAVFQAKKRVLEPFGLVEEGAK
Example of AAV6 VP1u (6VP1u) capsid protein
sequence
(SEQ ID NO: 6)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPFGLVEEGAK
Example of AAV7 VP1u (7VP1u) capsid protein
sequence
(SEQ ID NO: 7)
MAADGYLPDWLEDNLSEGIREWWDLKPGAPKPKANQQKQDNGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAK
Example of AAV8 VP1u (8VP1u) capsid protein
sequence
(SEQ ID NO: 8)
MAADGYLPDWLEDNLSEGIREWWALKPGAPKPKANQQKQDDGRGLVLPGY
KYLGPFNGLDKGEPVNAADAAALEHDKAYDQQLQAGDNPYLRYNHADAEF
QERLQEDTSFGGNLGRAVFQAKKRVLEPLGIVEEGAK
Example of AAV9 VP1u (9VP1u) capsid protein
sequence
(SEQ ID NO: 9)
MAADGYLPDWLEDNLSEGIREWWALKPGAPQPKANQQHQDNARGLVLPGY
KYLGPGNGLDKGEPVNAADAAALEHDKAYDQQKAGDNPYLKYNHADAEFQ
ERLKEDTSFGGNLGRAVFQAKKRLLEPLGLVEEAAK
Example of AAVrh.10 VP1u (10VP1u) capsid protein
sequence
(SEQ ID NO: 10)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAK
Example of AAV11 VP1u (11VP1u) capsid protein
sequence
(SEQ ID NO: 11)
MAADGYLPDWLEDNLSEGIREWWDLKPGAPKPKANQQKQDDGRGLVLPGY
KYLGPFNGLDKGEPVNAADAAALEHDKAYDQQLKAGDNPYLRYNHADAEF
QERLQEDTSFGGNLGRAVFQAKKRVLEPLGLVEEGAK
Example of AAV12 VP1u (12VP1u) capsid protein
sequence
(SEQ ID NO: 12)
MAADGYLPDWLEDNLSEGIREWWALKPGAPQPKANQQHQDNGRGLVLPGY
KYLGPFNGLDKGEPVNEADAAALEHDKAYDKQLEQGDNPYLKYNHADAEF
QQRLATDTSFGGNLGRAVFQAKKRILEPLGLVEEGVK
Example of AAV13 VP1u (13VP1u) capsid protein
sequence
(SEQ ID NO: 13)
MTDGYLPDWLEDNLSEGVREWWALQPGAPKPKANQQHQDNARGLVLPGYK
YLGPGNGLDKGEPVNAADAAALEHDKAYDQQLKAGDNPYLKYNHADAEFQ
ERLQEDTSFGGNLGRAVFQAKKRILEPLGLVEEAAK

Non-limiting examples of wild-type VP1 capsid protein sequences are provided below.

| Example of AAV1 VP1 capsid protein sequence | |
|---|---|
| (SEQ ID NO: 14) | |
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPLG LVEEGAKTAP GKKRPVEQSP |
| 151 | QEPDSSSGIG KTGQQPAKKR LNFGQTGDSE SVPDPQPLGE PPATPAAVGP |
| 201 | TTMASGGGAP MADNNEGADG VGNASGNWHC DSTWLGDRVI TTSTRTWALP |
| 251 | TYNNHLYKQI SSASTGASND NHYFGYSTPW GYFDFNRFHC HFSPRDWQRL |
| 301 | INNNWGFRPK RLNFKLFNIQ VKEVTTNDGV TTIANNLTST VQVFSDSEYQ |
| 351 | LPYVLGSAHQ GCLPPFPADV FMIPQYGYLT LNNGSQAVGR SSFYCLEYFP |
| 401 | SQMLRTGNNF TESYTFEEVP FHSSYAHSQS LDRLMNPLID QYLYYLNRTQ |
| 451 | NQSGSAQNKD LLFSRGSPAG MSVQPKNWLP GPCYRQQRVS KTKTDNNNSN |
| 501 | FTWTGASKYN LNGRESIINP GTAMASHKDD EDKFFPMSGV MIFGKESAGA |
| 551 | SNTALDNVMI TDEEEIKATN PVATERFGTV AVNFQSSSTD PATGDVHAMG |
| 601 | ALPGMVWQDR DVYLQGPIWA KIPHTDGHFH PSPLMGGFGL KNPPPQILIK |
| 651 | NTPVPANPPA EFSATKFASF ITQYSTGQVS VEIEWELQKE NSKRWNPEVQ |
| 701 | YTSNYAKSAN VDFTVDNNGL YTEPRPIGTR YLTRPL |
| Example of AAV2 VP1 capsid protein sequence | |
| (SEQ ID NO: 15) | |
| 1 | MAADGYLPDW LEDTLSEGIR QWWKLKPGPP PPKPAERHKD DSRGLVLPGY |
| 51 | KYLGPFNGLD KGEPVNEADA AALEHDKAYD RQLDSGDNPY LKYNHADAEF |
| 101 | QERLKEDTSF GGNLGRAVFQ AKKRVLEPLG LVEEPVKTAP GKKRPVEHSP |
| 151 | VEPDSSSGTG KAGQQPARKR LNFGQTGDAD SVPDPQPLGQ PPAAPSGLGT |
| 201 | NTMATGSGAP MADNNEGADG VGNSSGNWHC DSTWMGDRVI TTSTRTWALP |
| 251 | TYNNHLYKQI SSQSGASNDN HYFGYSTPWG YFDFNRFHCH FSPRDWQRLI |
| 301 | NNNWGFRPKR LNFKLFNIQV KEVTQNDGTT TIANNLTSTV QVFTDSEYQL |
| 351 | PYVLGSAHQG CLPPFPADVF MVPQYGYLTL NNGSQAVGRS SFYCLEYFPS |
| 401 | QMLRTGNNFT FSYTFEDVPF HSSYAHSQSL DRLMNPLIDQ YLYYLSRTNT |
| 451 | PSGTITQSRL QFSQAGASDI RDQSRNWLPG PCYRQQRVSK TSADNNNSEY |
| 501 | SWTGATKYHL NGRDSLVNPG PAMASHKDDE EKFFPQSGVL IFGKQGSEKT |
| 551 | NVDIEKVMIT DEEEIRTTNP VATEQYGSVS TNLQRGNRQA ATADVNTQGV |
| 601 | LPGMVWQDRD VYLQGPIWAK IPHTDGHFHP SPLMGGFGLK HPPPQILIKN |
| 651 | TPVPANPSTT FSAAKFASFI TQYSTGQVSV EIEWELQKEN SKRWNPEIQY |
| 701 | TSNYNKSVNV DFTVDTNGVY SEPRPIGTRY LTRNL |
| Example of AAV3 VP1 capsid protein sequence | |
| (SEQ ID NO: 16) | |
| 1 | MAADGYLPDW LEDNLSEGIR EWWALKPGVP QPKANQQHQD NRRGINLPGY |
| 51 | KYLGPGNGLD KGEPVNEADA AALEHDKAYD QQLKAGDNPY LKYNHADAEF |
| 101 | QERLQEDTSF GGNLGRAVFQ AKKRILEPLG LVEEAAKTAP GKKGAVDQSP |
| 151 | QEPDSSSGVG KSGKQPARKR LNFGQTGDSE SVPDPQPLGE PPAAPTSLGS |
| 201 | NTMASGGGAP MADNNEGADG VGNSSGNWHC DSQWLGDRVI TTSTRTWALP |
| 251 | TYNNHLYKQI SSQSGASNDN HYFGYSTPWG YFDFNRFHCH FSPRDWQRLI |
| 301 | NNNWGFRPKK LSFKLFNIQV RGVTQNDGTT TIANNLTSTV QVFTDSEYQL |
| 351 | PYVLGSAHQG CLPPFPADVF MVPQYGYLTL NNGSQAVGRS SFYCLEYFPS |
| 401 | QMLRTGNNFQ FSYTFEDVPH HSSYAHSQSL DRLMNPLIDQ YLYYLNRTQG |
| 451 | TTSGTTNQSR LLFSQAGPQS MSLQARNWLP GPCYRQQRLS KTANDNNNSN |
| 501 | FPWTAASKYH LNGRDSLVNP GPAMASHKDD EEKFFPMHGN LIFGKEGTTA |
| 551 | SNAELDNVMI TDEEEIRTTN PVATEQYGTV ANNLQSSNTA PTTGTVNHQG |
| 601 | ALPGMVWQDR DVYLQGPIWA KIPHTDGHFH PSPLMGGFGL KHPPPQIMIK |
| 651 | NTPVPANPPT TFSPAKFASF ITQYSTGQVS VEIEWELQKE NSKRWNPEIQ |
| 701 | YTSNYNKSVN VDFTVDTNGV YSEPRPIGTR YLTRNL |
| Example of AAV4 VP1 capsid protein sequence | |
| (SEQ ID NO: 17) | |
| 1 | MTDGYLPDWL EDNLSEGVRE WWALQPGAPK PKANQQHQDN ARGLVLPGYK |
| 51 | YLGPGNGLDK GEPVNAADAA ALEHDKAYDQ QLKAGDNPYL KYNHADAEFQ |
| 101 | QRLQGDTSFG GNLGRAVFQA KKRVLEPLGL VEQAGETAPG KKRPLIESPQ |
| 151 | QPDSSTGIGK KGKQPAKKKL VFEDETGAGD GPPEGSTSGA MSDDSEMRAA |
| 201 | AGGAAVEGGQ GADGVGNASG DWHCDSTWSE GHVTTTSTRT WVLPTYNNHL |
| 251 | YKRLGESLQS NTYNGFSTPW GYFDFNRFHC HFSPRDWQRL INNNWGMRPK |
| 301 | AMRVKIFNIQ VKEVTTSNGE TTVANNLTST VQIFADSSYE LPYVMDAGQE |
| 351 | GSLPPFPNDV FMVPQYGYCG LVTGNTSQQQ TDRNAFYCLE YFPSQMLRTG |
| 401 | NNFEITYSFE KVPFHSMYAH SQSLDRLMNP LIDQYLWGLQ STTTGTTLNA |
| 451 | GTATTNFTKL RPTNFSNFKK NWLPGPSIKQ QGFSKTANQN YKIPATGSDS |
| 501 | LIKYETHSTL DGRWSALTPG PPMATAGPAD SKFSNSQLIF AGPKQNGNTA |
| 551 | TVPGTLIFTS EEELAATNAT DTDMWGNLPG GDQSNSNLPT VDRLTALGAV |
| 601 | PGMVWQNRDI YYQGPIWAKI PHTDGHFHPS PLIGGFGLKH PPPQIFIKNT |
| 651 | PVPANPATTF SSTPVNSFIT QYSTGQVSVQ IDWEIQKERS KRWNPEVQFT |
| 701 | SNYGQQNSLL WAPDAAGKYT EPRAIGTRYL THHL |
| Example of AAV5 VP1 capsid protein sequence | |
| (SEQ ID NO: 18) | |
| 1 | MSFVDHPPDW LEEVGEGLRE FLGLEAGPPK PKPNQQHQDQ ARGLVLPGYN |
| 51 | YLGPGNGLDR GEPVNRADEV AREHDISYNE QLEAGDNPYL KYNHADAEFQ |
| 101 | EKLADDTSFG GNLGKAVFQA KKRVLEPFGL VEEGAKTAPT GKRIDDHFPK |
| 151 | RKKARTEEDS KPSTSSDAEA GPSGSQQLQI PAQPASSLGA DTMSAGGGGP |
| 201 | LGDNNQGADG VGNASGDWHC DSTWMGDRVV TKSTRTWVLP SYNNHQYREI |
| 251 | KSGSVDGSNA NAYFGYSTPW GYFDFNRFHS HWSPRDWQRL INNYWGFRPR |
| 301 | SLRVKIFNIQ VKEVTVQDST TTIANNLTST VQVFTDDDYQ LPYVVGNGTE |
| 351 | GCLPAFPPQV FTLPQYGYAT LNRDNTENPT ERSSFFCLEY FPSKMLRTGN |
| 401 | NFEFTYNFEE VPFHSSFAPS QNLFKLANPL VDQYLYRFVS TNNTGGVQFN |
| 451 | KNLAGRYANT YKNWFPGPMG RTQGWNLGSG VNRASVSAFA TTNRMELEGA |
| 501 | SYQVPPQPNG MTNNLQGSNT YALENTMIFN SQPANPGTTA TYLEGNMLIT |
| 551 | SESETQPVNR VAYNVGGQMA TNNQSSTTAP ATGTYNLQEI VPGSVWMERD |
| 601 | VYLQGPIWAK IPETGAHFHP SPAMGGFGLK HPPPMMLIKN TPVPGNITSF |
| 651 | SDVPVSSFIT QYSTGQVTVE MEWELKKENS KRWNPEIQYT NNYNDPQFVD |
| 701 | FAPDSTGEYR TTRPIGTRYL TRPL |
| Example of AAV6 VP1 capsid protein sequence | |
| (SEQ ID NO: 19) | |
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPFG LVEEGAKTAP GKKRPVEQSP |
| 151 | QEPDSSSGIG KTGQQPAKKR LNFGQTGDSE SVPDPQPLGE PPATPAAVGP |
| 201 | TTMASGGGAP MADNNEGADG VGNASGNWHC DSTWLGDRVI TTSTRTWALP |
| 251 | TYNNHLYKQI SSASTGASND NHYFGYSTPW GYFDFNRFHC HFSPRDWQRL |
| 301 | INNNWGFRPK RLNFKLFNIQ VKEVTTNDGV TTIANNLTST VQVFSDSEYQ |
| 351 | LPYVLGSAHQ GCLPPFPADV FMIPQYGYLT LNNGSQAVGR SSFYCLEYFP |
| 401 | SQMLRTGNNF TFSYTFEDVP FHSSYAHSQS LDRLMNPLID QYLYYLNRTQ |
| 451 | NQSGSAQNKD LLFSRGSPAG MSVQPKNWLP GPCYRQQRVS KTKTDNNNSN |
| 501 | FTWTGASKYN LNGRESIINP GTAMASHKDD KDKFFPMSGV MIFGKESAGA |
| 551 | SNTALDNVMI TDEEEIKATN PVATERFGTV AVNLQSSSTD PATGDVHVMG |
| 601 | ALPGMVWQDR DVYLQGPIWA KIPHTDGHFH PSPLMGGFGL KHPPPQILIK |
| 651 | NTPVPANPPA ERSATKFASF ITQYSTGQVS VEIEWELQKE NSKRWNPEVQ |
| 701 | YTSNYAKSAN VDFTVDNNGL YTEPRPIGTR YLTRPL |
| Example of AAV7 VP1 capsid protein sequence | |
| (SEQ ID NO: 20) | |
| 1 | MAADGYLPDW LEDNLSEGIR EWWDLKPGAP KPKANQQKQD NGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVLEPLG LVEEGAKTAP AKKRPVEPSP |
| 151 | QRSPDSSTGI GKKGQQPARK RLNFGQTGDS ESVPDPQPLG EPPAAPSSVG |
| 201 | SGTVAAGGGA PMADNNEGAD GVGNASGNWH CDSTWLGDRV ITTSTRTWAL |
| 251 | PTYNNHLYKQ ISSETAGSTN DNTYFGYSTP WGYFDFNRFH CHFSPRDWQR |
| 301 | LINNNWGFRP KKLRFKLFNI QVKEVTTNDG VTTIANNLTS TIQVFSDSEY |
| 351 | QLPYVLGSAH QGCLPPFPAD VFMIPQYGYL TLNNGSQSVG RSSFYCLEYF |
| 401 | PSQMLRTGNN FEFSYSFEDV PFHSSYAHSQ SLDRLMNPLI DQYLYYLART |
| 451 | QSNPGGTAGN RELQFYQGGP STMAEQAKNW LPGPCFRQQR VSKTLDQNNN |
| 501 | SNFAWTGATK YHLNGRNSLV NPGVAMATHK DDEDRFFPSS GVLIFGKTGA |
| 551 | TNKTTLENVL MTNEEEIRPT NPVATEEYGI VSSNLQAANT AAQTQVVNNQ |
| 601 | GALPGMVWQN RDVYLQGPIW AKIPHTDGNF HPSPLMGGFG LKHPPPQILI |
| 651 | KNTPVPANPP EVFTPAKFAS FITQYSTGQV SVEIEWELQK ENSKRWNPEI |
| 701 | QYTSNFEKQT GVDFAVDSQG VYSEPRPIGT RYLTRNL |
| Example of AAV8 VP1 capsid protein sequence | |
| (SEQ ID NO: 21) | |
| 1 | MAADGYLPDW LEDNLSEGIR EWWALKPGAP KPKANQQKQD DGRGLVLPGY |
| 51 | KYLGPFNGLD KGEPVNAADA AALEHDKAYD QQLQAGDNPY LRYNHADAEF |
| 101 | QERLQEDTSF GGNLGRAVFQ AKKRVLEPLG LVEEGAKTAP GKKRPVEPSP |
| 151 | QRSPDSSTGI GKKGQQPARK RLNFGQTGDS ESVPDPQPLG EPPAAPSGVG |
| 201 | PNTMAAGGGA PMADNNEGAD GVGSSSGNWH CDSTWLGDRV ITTSTRTWAL |
| 251 | PTYNNHLYKQ ISNGTSGGAT NDNTYFGYST PWGYFDFNRF HCHFSPRDWQ |
| 301 | RLINNNWGFR PKRLSFKLFN IQVKEVTQNE GTKTIANNLT STIQVFTDSE |
| 351 | YQLPYVLGSA HQGCLPPFPA DVFMIPQYGY LTLNNGSQAV GRSSFYCLEY |
| 401 | FPSQMIRTGN NFQFTYTFED VPFHSSYAHS QSLDRLMNPL IDQYLYYLSR |
| 451 | TQTTGGTANT QTLGFSQGGP NTMANQAKNW LPGPCYRQQR VSTTTGQNNN |
| 501 | SNFAWTAGTK YHLNGRNSLA NPGIAMATHK DDEERFFPSN GILIFGKQNA |
| 551 | ARDNADYSDV MLTSEEEIKT TNPVATEEYG IVADNLQQQN TAPQIGTVNS |
| 601 | QGALPGMVWQ NRDVYLQGPI WAKIPHTDGN FHPSPLMGGF GLKHPPPQIL |
| 651 | IKNTPVPADP PTTFNQSKLN SFITQYSTGQ VSVEIEWELQ KENSKRWNPE |
| 701 | IQYTSNYYKS TSVDFAVNTE GVYSEPRPIG TRYLTRNL |
| Example of AAV9 VP1 capsid protein sequence | |
| (SEQ ID NO: 22) | |
| 1 | MAADGYLPDW LEDNLSEGIR EWWALKPGAP QPKANQQHQD NARGLVLPGY |
| 51 | KYLGPGNGLD KGEPVNAADA AALEHDKAYD QQLKAGDNPY LKYNHADAEF |
| 101 | QERLKEDTSF GGNLGRAVFQ AKKRLLEPLG LVEEAAKTAP GKKRPVEQSP |
| 151 | QEPDSSAGIG KSGAQPAKKR LNFGQTGDTE SVPDPQPIGE PPAAPSGVGS |
| 201 | LTMASGGGAP VADNNEGADG VGSSSGNWHC DSQWLGDRVI TTSTRTWALP |
| 251 | TYNNHLYKQI SNSTSGGSSN DNAYFGYSTP WGYFDFNRFH CHFSPRDWQR |
| 301 | LINNNWGFRP KRLNTKLFNI QVKEVTDNNG VKTIANNLTS TVQVFTDSDY |
| 351 | QLPYVLGSAH EGCLPPFPAD VFMIPQYGYL TLNDGSQAVG RSSFYCLEYF |
| 401 | PSQMLRTGNN FQFSYEFENV PFHSSYAHSQ SLDRLMNPLI DQYLYYLSKT |
| 451 | INGSGQNQQT LKFSVAGPSN MAVQGRNYIP GPSYRQQRVS TTVTQNNNSE |
| 501 | FAWPGASSWA LNGRNSLMNP GPAMASHKEG EDRFFPLSGS LIFGKQGTGR |
| 551 | DNVDADKVMI TNEEEIKTTN PVATESYGQV ATNHQSAQAQ AQTGWVQNQG |
| 601 | ILPGMVWQDR DVYLQGPIWA KIPHTDGNFH PSPLMGGFGM KHPPPQILIK |
| 651 | NTPVPADPPT AFNKDKLNSF ITQYSTGQVS VEIEWELQKE NSKRWNPEIQ |
| 701 | YTSNYYKSNN VEFAVNTEGV YSEPRPIGTR YLTRNL |
| Example of AAV10 VP1 capsid protein sequence | |
| (SEQ ID NO: 23) | |
| 1 | MAADGYLPDW LEDNLSEGIR EWWDLKPGAP KPKANQQKQD DGRGLVLPGY |
| 51 | KYLGPFNGLD KGEPVNAADA AALEHDKAYD QQLKAGDNPY LRYNHADAEF |
| 101 | QERLQEDTSF GGNLGRAVFQ AKKRVIEPLG LVEEGAKTAP GKKRPVEPSP |
| 151 | QRSPDSSTGI GKKGQQPAKK RLNFGQTGDS ESVPDPQPIG EPPAGPSGLG |
| 201 | SGTMAAGGGA PMADNNEGAD GVGSSSGNWH CDSTWLGDRV ITTSTRTWAL |
| 251 | PTYNNHLYKQ ISNGTSGGST NDNTYFGYST PWGYFDFNRF HCHFSPRDWQ |
| 301 | RLINNNWGFR PKRLNFKLFN IQVKEVTQNE GTKTIANNLT STIQVFTDSE |
| 351 | YQLPYVLGSA HQGCLPPFPA DVFMIPQYGY LTLNNGSQAV GRSSFYCLEY |
| 401 | FPSQMLRTGN NFEFSYQFED VPHISSYAHS QSLDRLMNPL IDQYLYYLSR |
| 451 | TQSTGGTAGT QQLLFSQAGP NNMSAQAKNW LPGPCYRQQR VSTTLSQNNN |
| 501 | SNFAWTGATK YHLNGRDSLV NPGVAMATHK DDEERITPSS GVLMFGKQGA |
| 551 | GKDNVDYSSV MLTSEEEIKT TNPVATEQYG VVADNLQQQN AAPIVGAVNS |
| 601 | QGALPGMVWQ NRDVYLQGPI WAKIPHTDGN FHPSPLMGGF GLKHPPPQIL |
| 651 | IKNTPVPADP PTTFSQAKLA SFITQYSTGQ VSVEIEWELQ KENSKRWNPE |
| 701 | IQYTSNYYKS TNVDFAVNTD GTYSEPRPIG TRYLTRNL |

Example of AAV11 VP1 capsid protein sequence
(SEQ ID NO: 24)
MAADGYLPDWLEDNLSEGIREWWDLKPGAPKPKANQQKQDDGRGLVLPGYKYLGPF
NGLDKGEPVNAADAAALEHDKAYDQQLKAGDNPYLRYNHADAEFQERLQEDTSFGGN
LGRAVFQAKKRVLEPLGLVEEGAKTAPGKKRPLESPQEPDSSSGIGKKGKQPARKRLNF
EEDTGAGDGPPEGSDTSAMSSDIEMRAAPGGNAVDAGQGSDGVGNASGDWHCDSTWS
EGKVTTTSTRTWVLPTYNNHLYLRLGTTSSSNTYNGFSTPWGYFDFNRFHCHFSPRDW
QRLINNNWGLRPKAMRVKIFNIQVKEVTTSNGETTVANNLTSTVQIFADSSYELPYVMD
AGQEGSLPPFPNDVFMVPQYGYCGIVTGENQNQTDRNAFYCLEYFPSQMLRTGNNFEM
AYNFEKVPFHSMYAHSQSLDRLMNPLLDQYLWHLQSTTSGETLNQGNAATTFGKIRSG
DFAFYRKNWLPGPCVKQQRFSKTASQNYKIPASGGNALLKYDTHYTLNNRWSNIAPGP
PMATAGPSDGDFSNAQLIFPGPSVTGNTTTSANNLLFTSEEEIAATNPRDTDMFGQIADN
NQNATTAPITGNVTAMGVLPGMVWQNRDIYYQGPIWAKIPHADGHFHPSPLIGGFGLK
HPPPQIFIKNTPVPANPATTFTAARVDSFITQYSTGQVAVQIEWEIEKERSKRWNPEVQFT
SNYGNQSSMLWAPDTTGKYTEPRVIGSRYLTNHL
Example of AAV12 VP1 capsid protein sequence
(SEQ ID NO: 25)
MAADGYLPDWLEDNLSEGIREWWALKPGAPQPKANQQHQPNGRGLVLPGYKYLGPF
NGLDKGEPVNEADAAALEHDKAYDKQLEQGDNPYLKYNHADAEFQQRLATDTSFGGN
LGRAVFQAKKRILEPLGLVEEGVKTAPGKKRPLEKTPNRPTNPDSGKAPAKKKQKDGEP
ADSARRTLDFEDSGAGDGPPEGSSSGEMSHDAEMRAAPGGNAVEAGQGADGVGNASG
DWHCDSTWSEGRVTTTSTRTWVLPTYNNHLYLRIGTTANSNTYNGFSTPWGYFDFNRF
HCHFSPRDWQRLINNNWGILRPKSMRVKIFNIQVKEVTTSNGETTVANNLTSTVQIFADS
TYELPYVMDAGQEGSFPPFPNDVFMVPQYGYCGVVTGKNQNQTDRNAFYCLEYFPSQ
MLRTGNNFEVSYQFEKVPFHSMYAHSQSLDRMMNPLLDQYLWHLQSTTTGNSLNQGT
ATTTYGKITTGDFAYYRKNWLPGACIKQQKFSKNANQNYKIPASGGDALLKYDTHTTL
NGRWSNMAPGPPMATAGAGDSDFSNSQLIFAGPNPSGNTTTSSNNLLFTSEEEIATTNPR
DTDMFGQIADNNQNATTAPHIANLDAMGIVPGGMVWQNRDIYYQGPIWAKVPHTDGHF
HPSPLMGGFGLKHPPPQIFIKNTPVPANPNTTFSAARINSFLTQYSTGQVAVQIDWEIQKE
HSKRWNPEVQFTSNYGTQNSMLWAPDNAGNYHELRAIGSRFLTHHL
Example of AAV13 VP1 capsid protein sequence
(SEQ ID NO: 26)
MTDGYLPDWLEDNLSEGVREWWALQPGAPKPKANQQHQDNARGLVLPGYKYLGPGN
GLDKGEPVNAADAAALEHDKAYDQQLKAGDNPYLKYNHADAEFQERLQEDTSFGGNL
GRAVFQAKKRILEPLGLVEEAAKTAPGKKRPVEQSPAEPDSSSGIGKSGQQPARKRLNF
GQTGDTESVPDPQPLGQPPAAPSGVGSTTMASGGGAPMADNNEGADGVGNSSGNWHC
DSQWLGDRVITTSTRTWALPTYNNHLYKQISSQSGATNDNHYFGYSTPWGYFDFNRFH
CHFSPRDWQRLINNNWGFRPKRLNFKLFNIQVKEVTQNDGTTTIANNLTSTVQVFTDSE
YQLPYVLGSAHQGCLPPFPADVFMVPQYGYLTLNNGSQAVGRSSFYCLEYFPSQMLRT
GNNFQFSYTFEDVPFHSSYAHSQSLDRLMNPLIDQYLYYLNRTQTASGTQQSRLLFSQA
GPTSMSLQAKNWLPGPCYRQQRLSKQANDNNNSNFPWTGATKYHLNGRDSLVNPGPA
MASHKDDKEKFFPMHGTLIFGKEGTNANNADLENVMITDEEEIRTTNPVATEQYGTVSN
NLQNSNAGPTTGTVNHQGALPGMVWQDRDVYLQGPIWAKIPHTDGHFHPSPLMGGFG
LKHPPPQIMIKNTPVPANPPTNFSAAKFASFITQYSTGQVSVEIEWELQKENSKRWNPEIQ
YTSNYNKSVNVDFTVDTNGVYSEPRPIGTRYLTRNL

Chimaeric rAAV Capsid Proteins

Provided herein is a chimeric rAAV capsid protein comprising a region of VP1u that is replaced by the corresponding amino acids of the VP1u of a serotype that is different from the backbone serotype of the chimeric rAAV capsid protein.

In some embodiments, a chimeric rAAV capsid protein as provided herein has a backbone of a first serotype and a region of VP1u of a second serotype. In some embodiments, the backbone of a first serotype is a serotype that constitutes majority of the amino acid sequence of a VP1 protein. For example, if the majority of a VP1 protein is made up of amino acid sequence of AAV1 (see e.g., SEQ ID NO: 14), then the backbone serotype of that VP1 protein is of serotype 1.

As provided herein, the first serotype of a chimeric VP1 capsid protein (i.e., the serotype of its backbone) is serotype 1, 2, 3, 4, 5, 6, 7, 8, 9, 10, 11, 12, or 13. In some embodiments, the first serotype, i.e., the serotype of the backbone of VP1u, is serotype 2, 5, or 9.

In some embodiments, the second serotype, i.e. the serotype of the region of VP1u that is different from the serotype of the first serotype is serotype 1, 2, 3, 4, 5, 6, 7, 8, 9, 10, 11, 12, or 13. In some embodiments, the second serotype is serotype 1 or 8. In some embodiments, a second serotype is serotype 1. In some embodiments, a second serotype is serotype 8.

In some embodiments, a first serotype (i.e., backbone serotype) is serotype 1 and the second serotype (i.e. the serotype of the region of VP1u that is different from the serotype of the first serotype is) is serotype 2, 3, 4, 5, 6, 7, 8, 9, 10, 11, 12, or 13. In some embodiments, a first serotype (i.e., backbone serotype) is serotype 2 and the second serotype (i.e. the serotype of the region of VP1u that is different from the serotype of the first serotype is) is serotype 1, 3, 4, 5, 6, 7, 8, 9, 10, 11, 12, or 13. In some embodiments, a first serotype (i.e., backbone serotype) is serotype 3 and the second serotype (i.e. the serotype of the region of VP1u that is different from the serotype of the first serotype is) is serotype 1, 2, 4, 5, 6, 7, 8, 9, 10, 11, 12, or 13. In some embodiments, a first serotype (i.e., backbone serotype) is serotype 4 and the second serotype (i.e. the serotype of the region of VP1u that is different from the serotype of the first serotype is) is serotype 1, 2, 3, 5, 6, 7, 8, 9, 10, 11, 12, or 13. In some embodiments, a first serotype (i.e., backbone serotype) is serotype 5 and the second serotype (i.e. the serotype of the region of VP1u that is different from the serotype of the first serotype is) is serotype 1, 2, 3, 4, 6, 7, 8, 9, 10, 11, 12, or 13. In some embodiments, a first serotype (i.e., backbone serotype) is serotype 6 and the second serotype (i.e. the serotype of the region of VP1u that is different from the serotype of the first serotype is) is serotype 1, 2, 3, 4, 5, 7, 8, 9, 10, 11, 12, or 13. In some embodiments, a first serotype (i.e., backbone serotype) is serotype 7 and the second serotype (i.e. the serotype of the region of VP1u that is different from the serotype of the first serotype is) is serotype 1, 2, 3, 4, 5, 6, 8, 9, 10, 11, 12, or 13. In some embodiments, a first serotype (i.e., backbone serotype) is serotype 8 and the second serotype (i.e. the serotype of the region of VP1u that is different from the serotype of the first serotype is) is serotype 1, 2, 3, 4, 5, 6, 7, 9, 10, 11, 12, or 13. In some embodiments, a first serotype (i.e., backbone serotype) is serotype 9 and the second serotype (i.e. the serotype of the region of VP1u that is different from the serotype of the first serotype is) is serotype 1, 2, 3, 4, 5, 6, 7, 8, 10, 11, 12, or 13. In some embodiments, a first serotype (i.e., backbone serotype) is serotype 10 and the second serotype (i.e. the serotype of the region of VP1u that is different from the serotype of the first serotype is) is serotype 1, 2, 3, 4, 5, 6, 7, 8, 9, 11, 12, or 13. In some embodiments, a first serotype (i.e., backbone serotype) is serotype 11 and the second serotype (i.e. the serotype of the region of VP1u that is different from the serotype of the first serotype is) is serotype 1, 2, 3, 4, 5, 6, 7, 8, 9, 10, 12, or 13. In some embodiments, a first serotype (i.e., backbone serotype) is serotype 12 and the second serotype (i.e. the serotype of the region of VP1u that is different from the serotype of the first serotype is) is serotype 1, 2, 3, 4, 5, 6, 7, 8, 9, 10, 11, or 13. In some embodiments, a first serotype (i.e., backbone serotype) is serotype 13 and the second serotype (i.e. the serotype of the region of VP1u that is different from the serotype of the first serotype is) is serotype 1, 2, 3, 4, 5, 6, 7, 8, 9, 10, 11, or 12.

In some embodiments, the first serotype is serotype 2, 3, 4, 5, 6, 7, 8, 9, 10, 11, 12, or 13, and the second serotype is serotype 1. In some embodiments, the first serotype is serotype 1, 3, 4, 5, 6, 7, 8, 9, 10, 11, 12, or 13, and the second serotype is serotype 2. In some embodiments, the first serotype is serotype 1, 2, 4, 5, 6, 7, 8, 9, 10, 11, 12, or 13, and the second serotype is serotype 3. In some embodiments, the first serotype is serotype 1, 2, 3, 5, 6, 7, 8, 9, 10, 11, 12, or 13, and the second serotype is serotype 4. In some embodiments, the first serotype is serotype 1, 2, 3, 4, 6, 7, 8, 9, 10, 11, 12, or 13, and the second serotype is serotype 5. In some embodiments, the first serotype is serotype 1, 2, 3, 4, 5, 7, 8, 9, 10, 11, 12, or 13, and the second serotype is serotype 6. In some embodiments, the first serotype is serotype 1, 2, 3, 4, 5, 6, 8, 9, 10, 11, 12, or 13, and the second serotype is serotype 7. In some embodiments, the first serotype is serotype 1, 2, 3, 4, 5, 6, 7, 9, 10, 11, 12, or 13, and the second serotype is serotype 8. In some embodiments, the first serotype is serotype 1, 2, 3, 4, 5, 6, 7, 8, 10, 11, 12, or 13, and the second serotype is serotype 9. In some embodiments, the first serotype is serotype 1, 2, 3, 4, 5, 6, 7, 8, 9, 11, 12, or 13, and the second serotype is serotype 10. In some embodiments, the first serotype is serotype 1, 2, 3, 4, 5, 6, 7, 8, 9, 10, 12, or 13, and the second serotype is serotype 11. In some embodiments, the first serotype is serotype 1, 2, 3, 4, 5, 6, 7, 8, 9, 10, 11, or 13, and the second serotype is serotype 12. In some embodiments, the first serotype is serotype 1, 2, 3, 4, 5, 6, 7, 8, 9, 10, 11, or 12, and the second serotype is serotype 13.

In some embodiments, the first serotype is serotype 1, and the second serotype is serotype 8. In some embodiments, the first serotype is serotype 2, and the second serotype is serotype 1, or 8. In some embodiments, the first serotype is serotype 3, and the second serotype is serotype 1, or 8. In some embodiments, the first serotype is serotype 4, and the second serotype is serotype 1, or 8. In some embodiments, the first serotype is serotype 5, and the second serotype is serotype 1, or 8. In some embodiments, the first serotype is serotype 6, and the second serotype is serotype 1, or 8. In some embodiments, the first serotype is serotype 7, and the second serotype is serotype 1, or 8. In some embodiments, the first serotype is serotype 8, and the second serotype is serotype 1. In some embodiments, the first serotype is serotype 9, and the second serotype is serotype 1, or 8. In some embodiments, the first serotype is serotype 10, and the second serotype is serotype 1, or 8. In some embodiments, the first serotype is serotype 11, and the second serotype is serotype 1, or 8. In some embodiments, the first serotype is serotype 12, and the second serotype is serotype 1, or 8. In some embodiments, the first serotype is serotype 13, and the second serotype is serotype 1, or 8.

In some embodiments, the first serotype is serotype 2, and the second serotype is serotype 1. In some embodiments, the first serotype is serotype 2, and the second serotype is serotype 8. In some embodiments, the first serotype is serotype 5, and the second serotype is serotype 1. In some embodiments, the first serotype is serotype 5, and the second serotype is serotype 8. In some embodiments, the first serotype is serotype 9, and the second serotype is serotype 1. In some embodiments, the first serotype is serotype 9, and the second serotype is serotype 8.

In some embodiments, a VP1u of an rAAV capsid protein comprises regions of two or more different serotypes compared to the backbone serotype. For example, a VP1u of backbone serotype 1 can have a region of serotype 8 and a region of serotype 2.

In some embodiments, the region of VP1u of a second serotype is at least 5 amino acids long (e.g., at least 5, at least 6, at least 10, at least 20, at least 50, at least 100, or at least 130 amino acids long). In some embodiments, the region of VP1u of a second serotype is at least 50 amino acids long (e.g., at least 50, at least 70, at least 80, at least 100, or at least 130 amino acids long). In some embodiments, the region of VP1u of a second serotype is at least 100 amino acids long (e.g., at least 100, at least 110, at least 120, or at least 130 amino acids long).

In some embodiments, the region of VP1u that is of the second serotype is the entire VP1u (e.g., 137 amino acids of the N-term of VP1 of AAV1, AAV2, AAV3, AAV6, AAV7, AAV8, AAV9, AAV10, AAV11, or AAV12; or 136 amino acids of the N-term of VP1 of AAV4, AAV5, or AAV13). In some embodiments, a chimeric VP1 capsid protein has an entire VP1u of a second serotype that is different from the first serotype. The entire VP1u can be of serotype 1, 2, 3, 4, 5, 6, 7, 8, 9, rh.10, 11, 12, or 13. In some embodiments, a chimeric VP1 capsid protein has an entire VP1u of serotype 1. In some embodiments, a chimeric VP1 capsid protein has an entire VP1u of serotype 8. In some embodiments, a chimeric VP1 capsid protein as disclosed herein has an entire VP1u of any one of SEQ ID NOs: 1-13. In some embodiments, the region of VP1u of the second serotype is amino acids 1-137 of AAV1, AAV2, AAV3, AAV6, AAV7, AAV8, AAV9, AAVrh.10, AAV11, or AAV12. In some embodiments, the region of VP1u of the second serotype is amino acids 1-136 of AAV4, AAV5, or AAV13. In some embodiments, a chimeric VP1 capsid protein as disclosed herein has an entire VP1u of AAV1, and is SEQ ID NO: 1. In some embodiments, a chimeric VP1 capsid protein as disclosed herein has an entire VP1u of AAV8, and is SEQ ID NO: 8.

A non-limiting example of a sequence of a chimeric VP1 capsid protein with a backbone of serotype 5 and a VP1u of serotype 1 (AAV5-1VP1u) is SEQ ID NO: 27. A non-limiting example of a sequence of a chimeric VP1 capsid protein with a backbone of serotype 5 and a VP1u of serotype 8 (AAV5-8VP1u) is SEQ ID NO: 28. A non-limiting example of a sequence of a chimeric VP1 capsid protein with a backbone of serotype 9 and a VP1u of serotype 1 (AAV9-8VP1u) is SEQ ID NO: 29. A non-limiting example of a sequence of a chimeric VP1 capsid protein with a backbone of serotype 9 and a VP1u of serotype 8 (AAV9-8VP1u) is SEQ ID NO: 30. A non-limiting example of a sequence of a chimeric VP1 capsid protein with a backbone of serotype 8 and a VP1u of serotype 1 (AAV8-1VP1u) is SEQ ID NO: 41.

Example of amino acid sequence of chimeric VP1 capsid protein with
a backbone of AAV5 and VP1u of AAV1 (AAV5-1VP1u)
(SEQ ID NO: 27)
MAADGYLPDWLEDNLSEGIREWWDLKPGAPKPKANQQKQDDGRGLVITGYKYLGPF
NGLDKGEPVNAADANALEHDKAYDQQLKAGDNPYLRYNHADAEFQERLQEDTSFGGN
LGRAVFQAKKRVLEPLGLVEEGAKTAPTGKRIDDHFPKRKKARTEEDSKPSTSSDAEAG
PSGSQQLQIPAQPASSLGADTMSAGGGGPLGDNNQGADGVGNASGDWHCDSTWMGD
RVVTKSTRTWVLPSYNNHQYREIKSGSVDGSNANANTGYSTPWGYFDFNRFHSHWSPR
DWQRLINNYWGFRPRSLRVKIFNIQVKEVTVQDSTTTIANNLTSTVQVFTDDDYQLPYV
VGNGTEGCLPAFPPQVFTLPQYGYATLNRDNTENPTERSSFFCLEYFPSKMLRTGNNFEF
TYNFEEVPFHSSFAPSQNLFKLANPLVDQYLYRFVSTNNTGGVQFNKNLAGRYANTYK
NWFPGPMGRTQGWNLGSGVNRASVSAFATTNRMELEGASYQVPPQPNGMTNNLQGSN
TYALENTMIFNSQPANPGTTATYLEGNMLITSESETQPVNRVAYNVGGQMATNNQSSTT
APATGTYNLQEIVPGSVWMERDVYLQGPIWAKIPETGAHFHPSPAMGGFGLKHPPPMM
LIKNTPVPGNITSFSDVPVSSFITQYSTGQVTVEMEWELKKENSKRWNPEIQYTNNYNDP
QFVDFAPDSTGEYRTTRPIGTRYLTRPL
Example of amino acid sequence of chimeric VP1 capsid protein with
a backbone of AAV5 and VP1u of AAV8 (AAV5-8VP1u)
(SEQ ID NO: 28)
MAADGYLPDWLEDNLSEGIREWWALKPGAPKPKANQQKQDDGRGLVLPGYKYLGPF
NGLDKGEPVNAADAAALEHDKAYDQQLQAGDNPYLRYNHADAEFQERLQEDTSFGGN
LGRAVFQAKKRVLEPLGLVEEGAKTAPTGKRIDDHFPKRKKARTEEDSKPSTSSDAEAG
PSGSQQLQIPAQPASSLGADTMSAGGGGPLGDNNQGADGVGNASGDWHCDSTWMGD
RVVTKSTRTWVLPSYNNHQYREIKSGSVDGSNANAYFGYSTPWGYFDFNRFHSHWSPR
DWQRLINNYWGFRPRSLRVKIFNIQVKEVTVQDSTTTIANNLTSTVQVFTDDDYQLPYV
VGNGTEGCLPAFPPQVFTLPQYGYATLNRDNTENPTERSSFFCLEYFPSKMLRTGNNFEF
TYNFEEVPHASSFAPSQNLFKLANPLVDQYLYRFVSTNNTGGVQFNKNLAGRYANTYK
NWFPGPMGRTQGWNLGSGVNRASVSAFATTNRMELEGASYQVPPQPNGMTNNLQGSN
TYALENTMIFNSQPANPGTTATYLEGNMLITSESETQPVNRVAYNVGGQMATNNQSSTT
APATGTYNLQEIVPGSVWMERDVYLQGPIWAKIPETGAHFHPSPAMGGFGLKHPPPMM
LIKNTPVPGNITSFSDVPVSSFITQYSTGQVTVEMEWELKKENSKRWNPEIQYTNNYNDP
QFVDFAPDSTGEYRTTRRIGTRYLTRPL
Example of amino acid sequence of chimeric VP1 capsid protein with
a backbone of AAV9 and VP1u of AAV1 (AAV9-1VP1u)
(SEQ ID NO: 29)
MAADGYLPDWLEDNLSEGIREWWDLKPGAPKPKANQQKQDDGRGLVLPGYKYLGPF
NGLDKGEPVNAADAAALEHDKAYDQQLKAGDNPYLRYNHADAEFQERLQEDTSFGGN
LGRAVFQAKKRVLEPLGLVEEGAKTAPGKKRPVEQSPQEPDSSAGIGKSGAQPAKKRLN
FGQTGDTESVPDPQPIGEPPAAPSGVGSLTMASGGGAPVADNNEGADGVGSSSGNWHC
DSQWLGDRVITTSTRTWALPTYNNHLYKQISNSTSGGSSNDNAYFGYSTPWGYFDFNRF
HCHFSPRDWQRLNNNWGFRPKRLNFKLFNIQVKEVTDNNGVKTIANNLTSTVQVFTDS
DYQLPYVLGSAHEGCLPPFPADVFMIPQYGYLTLNDGSQAVGRSSFYCLEYFPSQMLRT
GNNFQFSYEFENVPFHSSYAHSQSLDRLMNPLIDQYLYYLSKTINGSGQNQQTLKFSVA
GPSNMAVQGRNYIPGPSYRQQRVSTTVTQNNNSEFAWPGASSWALNGRNSLMNPGPA
MASHKEGEDRFFPLSGSLIFGKQGTGRDNVDADKVMITNEEEIKTTNPVATESYGQVAT
NHQSAQAQAQTGWVQNQGILPGMVWQDRDVYLQGPIWAKIPHTDGNFHPSPLMGGFG
MKHPPPQILIKNTPVPADPPTAFNKDKLNSFITQYSTGQVSVEIEWELQKENSKRWNPEI
QYTSNYYKSNNVEFAVNTEGVYSEPRPIGTRYLTRNL
Example of amino acid sequence of chimeric VP1 capsid protein with
a backbone of AAV9 and VP1u of AAV8 (AAV9-8VP1u)
(SEQ ID NO: 30)
MAADGYLPDWLEDNLSEGIREWWALKPGAPKPKANQQKQDDGRGLVLPGYKYLGPF
NGLDKGEPVNAADAAALEHDKAYDQQLQAGDNPYLRYNHADAEFQERLQEDTSFGGN
LGRAVFQAKKRVLEPLGLVEEGAKTAPGKKRPVEQSPQEPDSSAGIGKSGAQPAKKRLN
FGQTGDTESVPDPQPIGEPPAAPSGVGSLTMASGGGAPVADNNEGADGVGSSSGNWHC
DSQWLGDRVITTSTRTWALPTYNNHLYKQISNSTSGGSSNDNAYFGYSTPWGYFDFNRF
HCHFSPRDWQRLINNNWGFRPKRLNFKLFNIQVKEVTDNNGVKTIANNLTSTVQVFTDS
DYQLPYVLGSAHEGCLPPFPADVFMIPQYGYLTLNDGSQAVGRSSFYCLEYFPSQMLRT
GNNFQFSYEFENVPFHSSYAHSQSLDRLMNPLIDQYLYYLSKTINGSGQNQQTLKFSVA
GPSNMAVQGRNYIPGPSYRQQRVSTTVTQNNNSEFAWPGASSWALNGRNSLMNPGPA
MASHKEGEDRFFPLSGSLIFGKQGTGRDNVDADKVMITNEEEIKTTNPVATESYGQVAT
NHQSAQAQAQTGWVQNQGILPGMVWQDRDVYLQGPIWAKIPHTDGNFHPSPLMGGFG
MKHPPPQILIKNTPVPADPPTAFNKDKLNSFITQYSTGQVSVEIEWELQKENSKRWNPEI
QYTSNYYKSNNVEFAVNTEGVYSEPRPIGTRYLTRNL
Example of amino acid sequence of chimeric VP1 capsid protein with
a backbone of AAV8 and VP1u of AAV1 (AAV8-1VP1u)
(SEQ ID NO: 41)
MAADGYLPDWLEDNLSEGIREWWDLKPGAPKPKANQQKQDDGRGLVLPGYKYLGPF
NGLDKGEPVNAADANALEHDKAYDQQLKAGDNPYLRYNHADAEFQERLQEDTSFGGN
LGRAVFQAKKRVLEPLGLVEEGAKTAPGKKRPVEPSPQRSPDSSTGIGKKGQQPARKRL
NFGQTGDSESVPDPQPLGEPPAAPSGVGPNTMAAGGGAPMADNNEGADGVGSSSGNW
HCDSTWLGDRVITTSTRTWALPTYNNHLYKQISNGTSGGATNDNTYFGYSTPWGYFDF
NRFHCHFSPRDWQRLINNNWGFRPKRLSFKLFNIQVKEVTQNEGTKTIANNLTSTIQVFT
DSEYQLPYVLGSAHQGCLPPFPADVFMIPQYGYLTLNNGSQAVGRSSFYCLEYFPSQML
RTGNNFQFTYTFEDVPHSSYAHSQSLDRLMNPLIDQYLYYLSRTQTTGGTANTQTLGF
SQGGPNTMANQAKNWLPGPCYRQQRVSTTTGQNNNSNFAWTAGTKYHLNGRNSLAN
PGIAMATHKDDEERFFPSNGILIFGKQNAARDNADYSDVKLTSEEEIKTTNPVATEEYGI
VADNLQQQNTAPIGTVNSQGALPGMVWQNRDVYLQGPIWAKIPHTDGNFHPSPLMG
GFGLKHPPPQILIKNTPVPADPPTTFNQSKLNSFITQYSTGQVSVEIEWELQKENSKRWNP
EIQYTSNYYKSTSVDFAVNTEGVYSEPRPIGTRYLTRNL

In some embodiments, the region of VP1u of a second serotype comprises amino acids that show poor conservation between sequences of various serotypes when aligned (see FIG. 3). In some embodiments, the region of VP1u of a second serotype comprises amino acids that are depicted as not showing either a “*,” a “.,“or a”:” in FIG. 3. In some embodiments, the region of VP1u of the second serotype comprises residues D24, K84, or both D24 and K84 of AAV1. In some embodiments, the region of VP1u of the second serotype comprises residues A24, Q84, or both A24 and Q84 of AAV8.

In some embodiments, the region of VP1u of the second serotype comprises a functional domain, or part of a function domain. A function domain is a series of amino acids that is either sufficient, necessary, or involved in the certain functions of the protein. For example, the phospholipase A2 domain in VP1u enables a virus particle comprising the protein to escape from the endocytic pathway during cellular trafficking and nuclear localization for delivery of encapsidated genetic material for replication. Other examples of functional domains within the VP1u of VP1 is the calcium binding site (YXGXG, SEQ ID NO: 34), A1 (123-KRVLEPLGL-131, SEQ ID NO: 35), and A69 (169-LNFGQTGDADSV-184, SEQ ID NO: 36) domains.

In some embodiments, the region of VP1u of the second serotype comprises a phospholipase A2 domain. The sequence of a phospholipase A2 domain is HDXXY (SEQ ID NO: 31). An example of a phospholipase A2 domain for AAV1 and 8 is HDKAY (SEQ ID NO: 32). An example of a phospholipase A2 domain for AAV5 is HDISY (SEQ ID NO: 33).

In some embodiments, a chimeric capsid protein as provided herein has a backbone of a first serotype, and more than one regions of VP1u of a second serotype. In some embodiments, a chimeric capsid protein as provided herein has a backbone of a first serotype, one or more regions of VP1u of a second serotype, and one or more regions of VP1u of a third serotype.

In some embodiments, a chimeric capsid protein as provided herein has a backbone of a first serotype, a region of VP1u of a second serotype, and one or more amino acid substitutions of a third serotype. In some embodiments, a chimeric capsid protein as provided herein further comprises one or more (e.g., 1, 2, 3, 4, 5, 6, 7, 8, 9, 10 or more) single amino acid substitutions (e.g., in the VP1u region and/or outside the VP1u region). Non-limiting examples single amino acid substitutions in the VP1u region are L129F, L129L, and F56G. In some embodiments, the second and third serotypes are the same. In some embodiments, the one or more amino acids substitutions are of a third serotype are made within the VP1u. In some embodiments, the one or more amino acids substitutions are of a third serotype are made outside the VP1u (e.g., in VP2, or VP3). In some embodiments, the one or more amino acids substitutions of a third serotype are in one or more of the following group of positions: L129, and F56. In some embodiments, the one or more amino acids substitutions of a third serotype are one or more of the following: L129F, L129L, and F56G.

In some embodiments, any one of the chimeric capsid proteins disclosed herein has a VP2/VP3 common region that is of a another serotype. For example, a chimeric capsid protein with the backbone of serotype 8 and VP1u of serotype 1 (AAV8-1VP1u) has a VP2/VP3 common region of serotype 2.

Non-limiting examples of chimeric rAAV proteins are provided in Table 1.

Chimaeric rAAV Particles

Provided herein are also rAAV particles that comprise any one of the chimeric VP1 capsid proteins disclosed herein. A rAAV particle may be an empty capsid, or may comprise a genetic load that is to be delivered to a cell.

Recombinant AAV particles may comprise a nucleic acid vector, which may comprise at a minimum: (a) one or more heterologous nucleic acid regions comprising a sequence encoding a protein or polypeptide of interest or an RNA of interest (e.g., a siRNA or microRNA), and (b) one or more regions comprising inverted terminal repeat (ITR) sequences (e.g., wild-type ITR sequences or engineered ITR sequences) flanking the one or more nucleic acid regions (e.g., heterologous nucleic acid regions). Herein, heterologous nucleic acid regions comprising a sequence encoding a protein of interest or RNA of interest are referred to as genes of interest.

In some embodiments, a gene of interest encodes a detectable molecule. In some embodiments, a detectable molecule is a fluorescent protein, a bioluminescent protein, or a protein that provides color (e.g., β-galactosidase, β-lactamases, β-glucuronidase and spheriodenone). In some embodiments, a detectable molecule is a fluorescent, bioluminescent or enzymatic protein or functional peptide or functional polypeptide thereof. In some embodiments, a gene of interest encodes a therapeutic protein or therapeutic RNA. In some embodiments, a therapeutic gene encodes an antibody, a peptibody, a growth factor, a clotting factor, a hormone, a membrane protein, a cytokine, a chemokine, an activating or inhibitory peptide acting on cell surface receptors or ion channels, a cell-permeant peptide targeting intracellular processes, a thrombolytic, an enzyme, a bone morphogenetic proteins, a nuclease or other protein used for gene editing, an Fc-fusion protein, an anticoagulant, a nuclease, guide RNA or other nucleic acid or protein for gene editing.

In some embodiments, the nucleic acid vector is between 4 kb and 5 kb in size (e.g., 4.2 to 4.7 kb in size). Any nucleic acid vector described herein may be encapsidated by a viral capsid, such as an AAV1, AAV2, AAV5, AAV8, or AAV9 capsid or any other serotype, which may comprise a chimeric capsid protein as described herein. In some embodiments, the nucleic acid vector is circular. In some embodiments, the nucleic acid vector is single-stranded. In some embodiments, the nucleic acid vector is double-stranded. In some embodiments, a double-stranded nucleic acid vector may be, for example, a self-complimentary vector that contains a region of the nucleic acid vector that is complementary to another region of the nucleic acid vector, initiating the formation of the double-strandedness of the nucleic acid vector.

Any one of the rAAV particles provided herein may have capsid proteins that have amino acids of different serotypes outside of the VP1u region. In some embodiments, the serotype of the backbone of the VP1 protein is different from the serotype of the ITRs and/or the Rep gene. In some embodiments, the serotype of the backbone of the VP1 capsid protein of a particle is the same as the serotype of the ITRs. In some embodiments, the serotype of the backbone of the VP1 capsid protein of a particle is the same as the serotype of the Rep gene.

Properties of a rAAV Particle Comprising a Chimeric VP, Capsid Protein and Methods of Measuring Improvement in Transduction/Infection

Compared to their wild-type counterparts, any one of the particles disclosed herein with chimeric VP1 capsid proteins shows a difference in one or more of the following properties: packaging efficiency, particle stability, antigenicity, rate of exposure of VP1u on the surface of the particle during endocytosis, tissue tropism, transduction efficiency, and infection efficiency. Transduction is the expression of the delivered genetic material in the target cells. Infection is the ability of the particle to enter a cell, replicate and be released, and infect other neighboring cells.

In some embodiments, a chimeric particle as disclosed herein shows an increased packaging efficiency, particle stability, antigenicity, rate of exposure of VP1u on the surface of the particle during endocytosis, transduction efficiency, and infection efficiency, compared to its wild-type counterpart. A wild-type counterpart is a particle which comprises elements that are the same as a chimeric particle (e.g., ITRs and rep gene), with the exception that it does not comprise the VP1u of a second serotype as that of the chimeric particle. In some embodiments, a chimeric particle as disclosed herein shows an increased transduction efficiency, compared to its wild-type counterpart.

Transduction efficiency can be measured by allowing rAAV particles of a fixed multiplicity of infection (MOI) to infect cells and measuring the amount of expressed RNA or protein from the genetic load that is delivered by the rAAV particles. For example, a luciferase gene can be delivered using rAAV particles and after a certain time (e.g., 24 h, or 48 h), either luciferase RNA expression or luciferase protein can be measured using one of numerous techniques known in the art (e.g., cell fractionation, polymerase chain reaction, or luciferase enzyme assays).

In some embodiments, a cell is in vivo (i.e., inside an organism). In some embodiments, a cell is an in vitro environment (e.g., in a tissue culture dish, or in a cultured organoid). A cell may be one of many cells cultured under certain conditions, or part of an organ that is harvested, part of an organoid, or an organism. In some embodiments, a cell disclosed herein is a eukaryotic cell (derived from a eukaryotic organism). In some embodiments, a eukaryotic cell is derived from ectoderm, endoderm, or mesoderm. In some embodiments, a eukaryotic cell derived from ectoderm is derived from surface ectoderm, neural crest or neural tube. In some embodiments, a eukaryotic cell derived from endoderm is derived from foregut, pharyngeal pouch (e.g., cells of thyroid gland or paraphyroid gland) or cloaca (e.g., urothelial cell). In some embodiments, a eukaryotic cell derived from mesoderm is derived from paraxial mesoderm, intermediate mesoderm (e.g., a renal cell or cell of the reproductive system) or lateral plate.

In some embodiments, a eukaryotic cell is a human cell. In some embodiments, a eukaryotic cell is a mouse cell, rat cell, cat cell, dog cell, hamster cell, or a cell from a non-human primate.

In some embodiments, a cell disclosed herein is a stem cell (e.g., an induced pluripotent stem cell). In some embodiments, a cell disclosed herein is immortalized (e.g. HEK293, A549, HeLa, Jurkat, 3T3, or Vero cell). A cell that is used to test the effect of replacing a region of VP1u as disclosed herein may be chosen based on the particular target tissue. For example, if infectivity of an rAAV particle is desired to be analyzed for the purpose of delivering a gene to the brain, a neuronal cell line (e.g., C6, or SH-SY5Y cells) may be infected. In some embodiments, a cell may be HEK293, HEPG2, LEC2, PRO5, ARPE19, or COS7 cells.

Nucleic Acids Encoding Chimeric rAAV Capsid Protein

Provided herein are nucleic acids that encode any one of the chimeric VP1 capsid proteins disclosed herein. A nucleic acid encoding a chimeric VP1 capsid protein can be used to encapsidate genetic material that is to be delivered to a cell, or make empty capsids. In some embodiments, a nucleic acid encoding a chimeric VP1 capsid protein is comprised in a nucleic acid vector. In some embodiments, the nucleic acid vector comprising nucleic acid encoding a chimeric VP1 protein is circular. In some embodiments, a nucleic acid vector is single-stranded.

In some embodiments, a nucleic acid vector is double-stranded. In some embodiments, a double-stranded nucleic acid vector may be, for example, a self-complimentary vector that contains a region of the nucleic acid vector that is complementary to another region of the nucleic acid vector, initiating the formation of the double-strandedness of the nucleic acid vector.

In some embodiments, an expression control element (e.g., a promoter) is also comprised by the nucleic acid that comprises the nucleic acid encoding chimeric VP1 protein such that they are operably linked. A discussion of expression control elements can be found below.

Examples of sequences of nucleic acids that encode chimeric VP1 proteins are provided in SEQ ID NOs: 37-40, and 41.

Example of nucleic acid sequence encoding chimeric VP1 capsid
protein with a backbone of AAV5 and VP1u of AAV1 (AAV5-1VP1u)
(SEQ ID NO: 37)
ATGGCTGCCGATGGTTATCTTCCAGATTGGCTCGAGGACAACCTCTCTGAGGGCATT
CGCGAGTGGTGGGACTTGAAACCTGGAGCCCCGAAGCCCAAAGCCAACCAGCAAAA
GCAGGACGACGGCCGGGGTCTGGTGCTTCCTGGCTACAAGTACCTCGGACCCTTCAA
CGGACTCGACAAGGGGGAGCCCGTCAACGCGGCGGACGCAGCGGCCCTCGAGCAC
GACAAGGCCTACGACCAGCAGCTCAAAGCGGGTGACAATCCGTACCTGCGGTATAA
CCACGCCGACGCCGAGTTTCAGGAGCGTCTGCAAGAAGATACGTCTTTTGGGGGCA
ACCTCGGGCGAGCAGTCTTCCAGGCCAAGAAGCGGGTTCTCGAACCTCTCGGTCTGG
TTGAGGAAGGCGCTAAGACGGCCCCTACCGGAAAGCGGATAGACGACCACTTTCCA
AAAAGAAAGAAGGCTCGGACCGAAGAGGACTCCAAGCCTTCCACCTCGTCAGACGC
CGAAGCTGGACCCAGCGGATCCCAGCAGCTGCAAATCCCAGCCCAACCAGCCTCAA
GTTTGGGAGCTGATACAATGTCTGCGGGAGGTGGCGGCCCATTGGGCGACAATAAC
CAAGGTGCCGATGGAGTGGGCAATGCCFCGGGAGATTGGCATTGCGATTCCACGTG
GATGGGGGACAGAGTCGTCACCAAGTCCACCCGAACCTGGGTGCTGCCCAGCTACA
ACAACCACCAGTACCGAGAGATCAAAAGCGGCTCCGTCGACGGAAGCAACGCCAAC
GCCTACTTTGGATACAGCACCCCCTGGGGGTACTTTGACTTTAACCGCTTCCACAGC
CACTGGAGCCCCCGAGACTGGCAAAGACTCATCAACAACTACTGGGGCTTCAGACC
CCGGTCCCTCAGAGTCAAAATCTTCAACATTCAAGTCAAAGAGGTCACGGTGCAGG
ACTCCACCACCACCATCGCCAACAACCTCACCTCCACCGTCCAAGTGTTTACGGACG
ACGACTACCAGCTGCCCTACGTCGTCGGCAACGGGACCGAGGGATGCCTGCCGGCC
TTCCCTCCGCAGGTCTTTACGCTGCCGCAGTACGGTTACGCGACGCTGAACCGCGAC
AACACAGAAAATCCCACCGAGAGGAGCAGCTTCTTCTGCCTAGAGTACTTTCCCAGC
AAGATGCTGAGAACGGGCAACAACTTTGAGTTTACCTACAACTTTGAGGAGGTGCC
CTTCCACTCCAGCTTCGCTCCCAGTCAGAACCTGTTCAAGCTGGCCAACCCGCTGGT
GGACCAGTACTTGTACCGCTTCGTGAGCACAAATAACACTGGCGGAGTCCAGTTCAA
CAAGAACCTGGCCGGGAGATACGCCAACACCTACAAAAACTGGTTCCCGGGGCCCA
TGGGCCGAACCCAGGGCTGGAACCTGGGCTCCGGGGTCAACCGCGCCAGTGTCAGC
GCCTTCGCCACGACCAATAGGATGGAGCTCGAGGGCGCGAGTTACCAGGTGCCCCC
GCAGCCGAACGGCATGACCAACAACCTCCAGGGCAGCAACACCTATGCCCTGGAGA
ACACTATGATCTTCAACAGCCAGCCGGCGAACCCGGGCACCACCGCCACGTACCTC
GAGGGCAACATGCTCATCACCAGCGAGAGCGAGACGCAGCCGGTGAACCGCGTGGC
GTACAACGTCGGCGGGCAGATGGCCACCAACAACCAGAGCTCCACCACTGCCCCCG
CGACCGGCACGTACAACCTCCAGGAAATCGTGCCCGGCAGCGTGTGGATGGAGAGG
GACGTGTACCTCCAAGGACCCATCTGGGCCAAGATCCCAGAGACGGGGGCGCACTT
TCACCCCTCTCCGGCCATGGGCGGATTCGGACTCAAACACCCACCGCCCATGATGCT
CATCAAGAACACGCCTGTGCCCGGAAATATCACCAGCTTCTCGGACGTGCCCGTCAG
CAGCTTCATCACCCAGTACAGCACCGGGCAGGTCACCGTGGAGATGGAGTGGGAGC
TCAAGAAGGAAAACTCCAAGAGGTGGAACCCAGAGATCCAGTACACAAACAACTA
CAACGACCCCCAGTTTGTGGACTTTGCCCCGGACAGCACCGGGGAATACAGAACCA
CCAGACCTATCGGAACCCGATACCTTACCCGACCCCTTTAATCTAGA
Example of nucleic acid sequence encoding chimeric VP1 capsid
protein with a backbone of AAV5 and VP1u of AAV8 (AAV5-8VP1u)
(SEQ ID NO: 38)
ATGGCTGCCGATGGTTATCTTCCAGATTGGCTCGAGGACAACCTCTCTGAGGGCATT
CGCGAGTGGTGGGCGCTGAAACCTGGAGCCCCGAAGCCCAAAGCCAACCAGCAAA
AGCAGGACGACGGCCGGGGTCTGGTGCTTCCTGGCTACAAGTACCTCGGACCCTTCA
ACGGACTCGACAAGGGGGAGCCCGTCAACGCGGCGGACGCAGCGGCCCTCGAGCA
CGACAAGGCCTACGACCAGCAGCTGCAGGCGGGTGACAATCCGTACCTGCGGTATA
ACCACGCCGACGCCGAGTTTCAGGAGCGTCTGCAAGAAGATACGTCTTTTGGGGGC
AACCTCGGGCGAGCAGTCTTCCAGGCCAAGAAGCGGGTTCTCGAACCTCTCGGTCTG
GTTGAGGAAGGCGCTAAGACGGCCCCTACCGGAAAGCGGATAGACGACCACTTTCC
AAAAAGAAAGAAGGCTCGGACCGAAGAGGACTCCAAGCCTTCCACCTCGTCAGACG
CCGAAGCTGGACCCAGCGGATCCCAGCAGCTGCAAATCCCAGCCCAACCAGCCTCA
AGTTTGGGAGCTGATACAATGTCTGCGGGAGGTGGCGGCCCATTGGGCGACAATAA
CCAAGGTGCCGATGGAGTGGGCAATGCCTCGGGAGATTGGCATTGCGATTCCACGT
GGATGGGGGACAGAGTCGTCACCAAGTCCACCCGAACCTGGGTGCTGCCCAGCTAC
AACAACCACCAGTACCGAGAGATCAAAAGCGGCTCCGTCGACGGAAGCAACGCCA
ACGCCTACTTTGGATACAGCACCCCCTGGGGGTACTTTGACTTTAACCGCTTCCACA
GCCACTGGAGCCCCCGAGACTGGCAAAGACTCATCAACAACTACTGGGGCTTCAGA
CCCCGGTCCCTCAGAGTCAAAATCTTCAACATTCAAGTCAAAGAGGTCACGGTGCA
GGACTCCACCACCACCATCGCCAACAACCTCACCTCCACCGTCCAAGTGTTTACGGA
CGACGACTACCAGCTGCCCTACGTCGTCGGCAACGGGACCGAGGGATGCCTGCCGG
CCTTCCCTCCGCAGGTCTTTACGCTGCCGCAGTACGGTTACGCGACGCTGAACCGCG
ACAACACAGAAAATCCCACCGAGAGGAGCAGCTTCTTCTGCCTAGAGTACTTTCCCA
GCAAGATGCTGAGAACGGGCAACAACTTTGAGTTTACCTACAACTTTGAGGAGGTG
CCCTTCCACTCCAGCTTCGCTCCCAGTCAGAACCTGTTCAAGCTGGCCAACCCGCTG
GTGGACCAGTACTTGTACCGCTTCGTGAGCACAAATAACACTGGCGGAGTCCAGTTC
AACAAGAACCTGGCCGGGAGATACGCCAACACCTACAAAAACTGGTTCCCGGGGCC
CATGGGCCGAACCCAGGGCTGGAACCTGGGCTCCGGGGTCAACCGCGCCAGTGTCA
GCGCCTTCGCCACGACCAATAGGATGGAGCTCGAGGGCGCGAGTTACCAGGTGCCC
CCGCAGCCGAACGGCATGACCAACAACCTCCAGGGCAGCAACACCTATGCCCTGGA
GAACACTATGATCTTCAACAGCCAGCCGGCGAACCCGGGCACCACCGCCACGTACC
TCGAGGGCAACATGCTCATCACCAGCGAGAGCGAGACGCAGCCGGTGAACCGCGTG
GCGTACAACGTCGGCGGGCAGATGGCCACCAACAACCAGAGCTCCACCACTGCCCC
CGCGACCGGCACGTACAACCTCCAGGAAATCGTGCCCGGCAGCGTGTGGATGGAGA
GGGACGTGTACCTCCAAGGACCCATCTGGGCCAAGATCCCAGAGACGGGGGCGCAC
TTTCACCCCTCTCCGGCCATGGGCGGATTCGGACTCAAACACCCACCGCCCATGATG
CTCATCAAGAACACGCCTGTGCCCGGAAATATCACCAGCTTCTCGGACGTGCCCGTC
AGCAGCTTCATCACCCAGTACAGCACCGGGCAGGTCACCGTGGAGATGGAGTGGGA
GCTCAAGAAGGAAAACTCCAAGAGGTGGAACCCAGAGATCCAGTACACAAACAAC
TACAACGACCCCCAGTTTGTGGACTTTGCCCCGGACAGCACCGGGGAATACAGAAC
CACCAGACCTATCGGAACCCGATACCTTACCCGACCCCTTTAA
Example of nucleic acid sequence encoding chimeric VP1 capsid
protein with a backbone of AAV9 and VP1u of AAV1 (AAV9-1VP1u)
(SEQ ID NO: 39)
ATGGCTGCCGATGGTTATCTTCCAGATTGGCTCGAGGACAACCTCTCTGAGGGCATT
CGCGAGTGGTGGGACTTGAAACCTGGAGCCCCGAAGCCCAAAGCCAACCAGCAAAA
GCAGGACGACGGCCGGGGTCTGGTGCTTCCTGGCTACAAGTACCTCGGACCCTTCAA
CGGACTCGACAAGGGGGAGCCCGTCAACGCGGCGGACGCAGCGGCCCTCGAGCAC
GACAAGGCCTACGACCAGCAGCTCAAAGCGGGTGACAATCCGTACCTGCGGTATAA
CCACGCCGACGCCGAGTTTCAGGAGCGTCTGCAAGAAGATACGTCTTTTGGGGGCA
ACCTCGGGCGAGCAGTCTTCCAGGCCAAGAAGCGGGTTCTCGAACCTCTCGGTCTGG
TTGAGGAAGGCGCTAAGACGGCTCCTGGAAAGAAGAGGCCTGTAGAGCAGTCTCCT
CAGGAACCGGACTCCTCCGCGGGTATTGGCAAATCGGGTGCACAGCCCGCTAAAAA
GAGACTCAATTTCGGTCAGACTGGCGACACAGAGTCAGTCCCAGACCCTCAACCAA
TCGGAGAACCTCCCGCAGCCCCCTCAGGTGTGGGATCTCTTACAATGGCTTCAGGTG
GTGGCGCACCAGTGGCAGACAATAACGAAGGTGCCGATGGAGTGGGTAGTTCCTCG
GGAAATTGGCATTGCGATTCCCAATGGCTGGGGGACAGAGTCATCACCACCAGCAC
CCGAACCTGGGCCCTGCCCACCTACAACAATCACCTCTACAAGCAAATCTCCAACAG
CACATCTGGAGGATCTTCAAATGACAACGCCTACTTCGGCTACAGCACCCCCTGGGG
GTATTTTGACTTCAACAGATTCCACTGCCACTTCTCACCACGTGACTGGCAGCGACT
CATCAACAACAACTGGGGATTCCGGCCTAAGCGACTCAACTTCAAGCTCTTCAACAT
TCAGGTCAAAGAGGTTACGGACAACAATGGAGTCAAGACCATCGCCAATAACCTTA
CCAGCACGGTCCAGGTCTTCACGGACTCAGACTATCAGCTCCCGTACGTGCTCGGGT
CGGCTCACGAGGGCTGCCTCCCGCCGTTCCCAGCGGACGTTTTCATGATTCCTCAGT
ACGGGTATCTGACGCTTAATGATGGAAGCCAGGCCGTGGGTCGTTCGTCCTTTTACT
GCCTGGAATATTTCCCGTCGCAAATGCTAAGAACGGGTAACAACTTCCAGTTCAGCT
ACGAGTTTGAGAACGTACCTTTCCATAGCAGCTACGCTCACAGCCAAAGCCTGGACC
GACTAATGAATCCACTCATCGACCAATACTTGTACTATCTCTCAAAGACTATTAACG
GTTCTGGACAGAATCAACAAACGCTAAAATTCAGTGTGGCCGGACCCAGCAACATG
GCTGTCCAGGGAAGAAACTACATACCTGGACCCAGCTACCGACAACAACGTGTCTC
AACCACTGTGACTCAAAACAACAACAGCGAATTTGCTTGGCCTGGAGCTTCTTCTTG
GGCTCTCAATGGACGTAATAGCTTGATGAATCCTGGACCTGCTATGGCCAGCCACAA
AGAAGGAGAGGACCGTTTCTTTCCTTTGTCTGGATCTTTAATTTTTGGCAAACAAGG
AACTGGAAGAGACAACGTGGATGCGGACAAAGTCATGATAACCAACGAAGAAGAA
ATTAAAACTACTAACCCGGTAGCAACGGAGTCCTATGGACAAGTGGCCACAAACCA
CCAGAGTGCCCAAGCACAGGCGCAGACCGGCTGGGTTCAAAACCAAGGAATACTTC
CGGGTATGGTTTGGCAGGACAGAGATGTGTACCTGCAAGGACCCATTTGGGCCAAA
ATTCCTCACACGGACGGCAACTTTCACCCTTCTCCGCTGATGGGAGGGTTTGGAATG
AAGCACCCGCCTCCTCAGATCCTCATCAAAAACACACCTGTACCTGCGGATCCTCCA
ACGGCCTTCAACAAGGACAAGCTGAACTCTTTCATCACCCAGTATTCTACTGGCCAA
GTCAGCGTGGAGATCGAGTGGGAGCTGCAGAAGGAAAACAGCAAGCGCTGGAACC
CGGAGATCCAGTACACTTCCAACTATTACAAGTCTAATAATGTTGAATTTGCTGTTA
ATACTGAAGGTGTATATAGTGAACCCCGCCCCATTGGCACCAGATACCTGACTCGTA
ATCTGTAATCTAGA
Example of nucleic acid sequence encoding chimeric VP1 capsid
protein with a backbone of AAV9 and VP1u of AAV8 (AAV9-8VP1u)
(SEQ ID NO: 40)
ATGGCTGCCGATGGTTATCTTCCAGATTGGCTCGAGGACAACCTCTCTGAGGGCATT
CGCGAGTGGTGGGCGCTGAAACCTGGAGCCCCGAAGCCCAAAGCCAACCAGCAAA
AGCAGGACGACGGCCGGGGTCTGGTGCTTCCTGGCTACAAGTACCTCGGACCCTTCA
ACGGACTCGACAAGGGGGAGCCCGTCAACGCGGCGGACGCAGCGGCCCTCGAGCA
CGACAAGGCCTACGACCAGCAGCTGCAGGCGGGTGACAATCCGTACCTGCGGTATA
ACCACGCCGACGCCGAGTTTCAGGAGCGTCTGCAAGAAGATACGTCTTTTGGGGGC
AACCTCGGGCGAGCAGTCTTCCAGGCCAAGAAGCGGGTTCTCGAACCTCTCGGTCTG
GTTGAGGAAGGCGCTAAGACGGCTCCTGGAAAGAAGAGGCCTGTAGAGCAGTCTCC
TCAGGAACCGGACTCCTCCGCGGGTATTGGCAAATCGGGTGCACAGCCCGCTAAAA
AGAGACTCAATTTCGGTCAGACTGGCGACACAGAGTCAGTCCCAGACCCTCAACCA
ATCGGAGAACCTCCCGCAGCCCCCTCAGGTGTGGGATCTCTTACAATGGCTTCAGGT
GGTGGCGCACCAGTGGCAGACAATAACGAAGGTGCCGATGGAGTGGGTAGTTCCTC
GGGAAATTGGCATTGCGATTCCCAATGGCTGGGGGACAGAGTCATCACCACCAGCA
CCCGAACCTGGGCCCTGCCCACCTACAACAATCACCTCTACAAGCAAATCTCCAACA
GCACATCTGGAGGATCTTCAAATGACAACGCCTACTTCGGCTACAGCACCCCCTGGG
GGTATTTTGACTTCAACAGATTCCACTGCCACTTCTCACCACGTGACTGGCAGCGAC
TCATCAACAACAACTGGGGATTCCGGCCTAAGCGACTCAACTTCAAGCTCTTCAACA
TTCAGGTCAAAGAGGTTACGGACAACAATGGAGTCAAGACCATCGCCAATAACCTT
ACCAGCACGGTCCAGGTCTTCACGGACTCAGACTATCAGCTCCCGTACGTGCTCGGG
TCGGCTCACGAGGGCTGCCTCCCGCCGTTCCCAGCGGACGTTTTCATGATTCCTCAG
TACGGGTATCTGACGCTTAATGATGGAAGCCAGGCCGTGGGTCGTTCGTCCTTTTAC
TGCCTGGAATATTTCCCGTCGCAAATGCTAAGAACGGGTAACAACTTCCAGTTCAGC
TACGAGTTTGAGAACGTACCTTTCCATAGCAGCTACGCTCACAGCCAAAGCCTGGAC
CGACTAATGAATCCACTCATCGACCAATACTTGTACTATCTCTCAAAGACTATTAAC
GGTTCTGGACAGAATCAACAAACGCTAAAATTCAGTGTGGCCGGACCCAGCAACAT
GGCTGTCCAGGGAAGAAACTACATACCTGGACCCAGCTACCGACAACAACGTGTCT
CAACCACTGTGACTCAAAACAACAACAGCGAATTTGCTTGGCCTGGAGCTTCTTCTT
GGGCTCTCAATGGACGTAATAGCTTGATGAATCCTGGACCTGCTATGGCCAGCCACA
AAGAAGGAGAGGACCGTTTCTTTCCTTTGTCTGGATCTTTAATTTTTGGCAAACAAG
GAACTGGAAGAGACAACGTGGATGCGGACAAAGTCATGATAACCAACGAAGAAGA
AATTAAAACTACTAACCCGGTAGCAACGGAGTCCTATGGACAAGTGGCCACAAACC
ACCAGAGTGCCCAAGCACAGGCGCAGACCGGCTGGGTTCAAAACCAAGGAATACTT
CCGGGTATGGTTTGGCAGGACAGAGATGTGTACCTGCAAGGACCCATTTGGGCCAA
AATTCCTCACACGGACGGCAACTTTCACCCTTCTCCGCTGATGGGAGGGTTTGGAAT
GAAGCACCCGCCTCCTCAGATCCTCATCAAAAACACACCTGTACCTGCGGATCCTCC
AACGGCCTTCAACAAGGACAAGCTGAACTCTTTCATCACCCAGTATTCTACTGGCCA
AGTCAGCGTGGAGATCGAGTGGGAGCTGCAGAAGGAAAACAGCAAGCGCTGGAAC
CCGGAGATCCAGTACACTTCCAACTATTACAAGTCTAATAATGTTGAATTTGCTGTT
AATACTGAAGGTGTATATAGTGAACCCCGCCCCATTGGCACCAGATACCTGACTCGT
AATCTGTAATCTAGA
Example of nucleic acid sequence encoding chimeric VP1 capsid
protein with a backbone of AAV8 and VP1u of AAV1 (AAV5-1VP1u)
(SEQ ID NO: 42)
ATGGCTGCCGATGGTTATCTTCCAGATTGGCTCGAGGACAACCTCTCTGAGGGCATT
CGCGAGTGGTGGGACTTGAAACCTGGAGCCCCGAAGCCCAAAGCCAACCAGCAAAA
GCAGGACGACGGCCGGGGTCTGGTGCTTCCTGGCTACAAGTACCTCGGACCCTTCAA
CGGACTCGACAAGGGGGAGCCCGTCAACGCGGCGGACGCAGCGGCCCTCGAGCAC
GACAAGGCCTACGACCAGCAGCTCAAAGCGGGTGACAATCCGTACCTGCGGTATAA
CCACGCCGACGCCGAGTTTCAGGAGCGTCTGCAAGAAGATACGTCTTTTGGGGGCA
ACCTCGGGCGAGCAGTCTTCCAGGCCAAGAAGCGGGTTCTCGAACCTCTCGGTCTGG
TTGAGGAAGGCGCTAAGACGGCTCCTGGAAAGAAGAGACCGGTAGAGCCATCACCC
CAGCGTTCTCCAGACTCCTCTACGGGCATCGGCAAGAAAGGCCAACAGCCCGCCAG
AAAAAGACTCAATTTTGGTCAGACTGGCGACTCAGAGTCAGTTCCAGACCCTGAACC
TCTCGGAGAACCTCCAGCAGCGCCCTCTGGTGTGGGACCTAATACAATGGCTGCAG
GCGGTGGCGCACCAATGGCAGACAATAACGAAGGCGCCGACGGAGTGGGTAGTTCC
TCGGGAAATTGGCATTGCGATTCCACATGGCTGGGCGACAGAGTCATCACCACCAG
CACCCGAACCTGGGCCCTGCCCACCTACAACAACCACCTCTACAAGCAAATCTCCAA
CGGGACATCGGGAGGAGCCACCAACGACAACACCTACTTCGGCTACAGCACCCCCT
GGGGGTATTTTGACTTTAACAGATTCCACTGCCACTTTTCACCACGTGACTGGCAGC
GACTCATCAACAACAACTGGGGATTCCGGCCCAAGAGACTCAGCTTCAAGCTCTTCA
ACATCCAGGTCAAGGAGGTCACGCAGAATGAAGGCACCAAGACCATCGCCAATAAC
CTCACCAGCACCATCCAGGTGTTTACGGACTCGGAGTACCAGCTGCCGTACGTTCTC
GGCTCTGCCCACCAGGGCTGCCTGCCTCCGTTCCCGGCGGACGTGTTCATGATTCCC
CAGTACGGCTACCTAACACTCAACAACGGTAGTCAGGCCGTGGGACGCTCCTCCTTC
TACTGCCTGGAATACTTTCCTTCGCAGATGCTGAGAACCGGCAACAACTTCCAGTTT
ACTTACACCTTCGAGGACGTGCCTTTCCACAGCAGCTACGCCCACAGCCAGAGCTTG
GACCGGCTGATGAATCCTCTGATTGACCAGTACCTGTACTACTTGTCTCGGACTCAA
ACAACAGGAGGCACGGCAAATACGCAGACTCTGGGCTTCAGCCAAGGTGGGCCTAA
TACAATGGCCAATCAGGCAAAGAACTGGCTGCCAGGACCCTGTTACCGCCAACAAC
GCGTCTCAACGACAACCGGGCAAAACAACAATAGCAACTTTGCCTGGACTGCTGGG
ACCAAATACCATCTGAATGGAAGAAATTCATTGGCTAATCCTGGCATCGCTATGGCA
ACACACAAAGACGACGAGGAGCGTTTTTTTCCCAGTAACGGGATCCTGATTTTTGGC
AAACAAAATGCTGCCAGAGACAATGCGGATTACAGCGATGTCATGCTCACCAGCGA
GGAAGAAATCAAAACCACTAACCCTGTGGCTACAGAGGAATACGGTATCGTGGCAG
ATAACTTGCAGCAGCAAAACACGGCTCCTCAAATTGGAACTGTCAACAGCCAGGGG
GCCTTACCCGGTATGGTCTGGCAGAACCGGGACGTGTACCTGCAGGGTCCCATCTGG
GCCAAGATTCCTCACACGGACGGCAACTTCCACCCGTCTCCGCTGATGGGCGGCTTT
GGCCTGAAACATCCTCCGCCTCAGATCCTGATCAAGAACACGCCTGTACCTGCGGAT
CCTCCGACCACCTTCAACCAGTCAAAGCTGAACTCTTTCATCACGCAATACAGCACC
GGACAGGTCAGCGTGGAAATTGAATGGGAGCTGCAGAAGGAAAACAGCAAGCGCT
GGAACCCCGAGATCCAGTACACCTCCAACTACTACAAATCTACAAGTGTGGACTTTG
CTGTTAATACAGAAGGCGTGTACTCTGAACCCCGCCCCATTGGCACCCGTTACCTCA
CCCGTAATCTGTAATCTAGA

Methods of Making Chimeric rAAV Particles

A rAAV particle or rAAV preparation containing such particles as disclosed herein may comprises a viral capsid and a nucleic acid vector, which is encapsidated by the viral capsid. As mentioned above, in some embodiments, the nucleic acid vector comprises (1) one or more heterologous nucleic acid regions comprising a sequence encoding an RNA, protein or polypeptide of interest, (2) one or more nucleic acid regions comprising a sequence that facilitates expression of the heterologous nucleic acid region (e.g., a promoter), and (3) one or more nucleic acid regions comprising a sequence that facilitate integration of the heterologous nucleic acid region (optionally with the one or more nucleic acid regions comprising a sequence that facilitates expression) into the genome of the subject. In some embodiments, viral sequences that facilitate integration comprise Inverted Terminal Repeat (ITR) sequences. In some embodiments, the nucleic acid vector comprises one or more heterologous nucleic acid regions comprising a sequence encoding an RNA, protein or polypeptide of interest operably linked to a control element (e.g., a promoter), wherein the one or more heterologous nucleic acid regions are flanked on each side with an ITR sequence. Such a nucleic acid vector is herein also referred to as AAV-ITR containing one or more genes of interest. The ITR sequences can be derived from any AAV serotype (e.g., serotype 1, 2, 3, 4, 5, 6, 7, 8, 9, 10, 11, 12, or 13) or can be derived from more than one serotype.

ITR sequences and plasmids containing ITR sequences are known in the art and commercially available (see, e.g., products and services available from Vector Biolabs, Philadelphia, Pa.; Cellbiolabs, San Diego, Calif.; Agilent Technologies, Santa Clara, Ca; and Addgene, Cambridge, Mass.; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J. Proc Natl Acad Sci USA. 1996 Nov. 26; 93(24):14082-7; and Curtis A. Machida. Methods in Molecular Medicine™. Viral Vectors for Gene TherapyMethods and Protocols. 10.1385/1-59259-304-6:201 © Humana Press Inc. 2003. Chapter 10. Targeted Integration by Adeno-Associated Virus. Matthew D. Weitzman, Samuel M. YoungJr., Toni Cathomen and Richard Jude Samulski; U.S. Pat. Nos. 5,139,941 and 5,962,313, all of which are incorporated herein by reference).

Genebank reference numbers for sequences of AAV serotypes 1, 2, 3, 3B, 4, 5, 6, 7, 8, 9, 10, 11, 12, and 13 are listed in patent publication WO2012064960, which is incorporated herein by reference in its entirety.

In some embodiments, the nucleic acid vector comprises one or more regions comprising a sequence that facilitates expression of the nucleic acid (e.g., the heterologous nucleic acid), e.g., expression control sequences operatively linked to the nucleic acid. Numerous such sequences are known in the art. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

To achieve appropriate expression levels of the protein or polypeptide of interest, any of a number of promoters suitable for use in the selected host cell may be employed. The promoter may be, for example, a constitutive promoter, tissue-specific promoter, inducible promoter, or a synthetic promoter.

For example, constitutive promoters of different strengths can be used. A nucleic acid vector described herein may include one or more constitutive promoters, such as viral promoters or promoters from mammalian genes that are generally active in promoting transcription. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A and cytomegalovirus (CMV) promoters. Non-limiting examples of constitutive mammalian promoters include various housekeeping gene promoters, as exemplified by the f-actin promoter (e.g. chicken f-actin promoter) and human elongation factor-1α (EF-1α) promoter.

Inducible promoters and/or regulatory elements may also be contemplated for achieving appropriate expression levels of the protein or polypeptide of interest. Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline.

Tissue-specific promoters and/or regulatory elements are also contemplated herein. Non-limiting examples of such promoters that may be used include airway epithelial cell-specific promoters.

Synthetic promoters are also contemplated herein. A synthetic promoter may comprise, for example, regions of known promoters, regulatory elements, transcription factor binding sites, enhancer elements, repressor elements, and the like.

The rAAV particle or particle within an rAAV preparation may be of any AAV serotype, including any derivative or pseudotype (e.g., 1, 2, 3, 4, 5, 6, 7, 8, 9, 10, 11, 12, 13, 2/1, 2/5, 2/8, 2/9, 3/1, 3/5, 3/8, or 3/9).

Pseudotyping refers to using the capsid of one serotype and the genome of another serotype, or the mixing of a capsid and genome from different viral serotypes. These serotypes are denoted using a slash, so that AAV2/5 indicates a virus containing the genome of serotype 2 packaged in the capsid from serotype 5.

As used herein, the serotype of an rAAV viral particle refers to the serotype of the capsid proteins of the recombinant virus. Non-limiting examples of derivatives and pseudotypes include rAAV2/1, rAAV2/5, rAAV2/8, rAAV2/9, AAV2-AAV3 hybrid. AAVrh.10, AAVhu.14, AAV3a/3b, AAVrh32.33, AAV-HSC15, AAV-HSC17, AAVhu.37, AAVrh.8, CHt-P6, AAV2.5, AAV6.2, AAV2i8, AAV-HSC15/17, AAVM41, AAV9.45, AAV6(Y445F/Y731F), AAV2.5T, AAV-HAE1/2, AAV clone 32/83, AAVShH10, AAV2 (Y->F), AAV8 (Y733F), AAV2.15, AAV2.4, AAVM41, and AAVr3.45. A non-limiting example of derivatives and pseudotypes that have chimeric VP1 proteins is rAAV2/5-1VP1u, which has the genome of AAV2, capsid backbone of AA5 and VP1u of AAV1. Other non-limiting example of derivatives and pseudotypes that have chimeric VP1 proteins are rAAV2/5-8VP1u, rAAV2/9-1VP1u, and rAAV2/9-8VP1u.

AAV derivatives/pseudotypes, and methods of producing such derivatives/pseudotypes are known in the art (see, e.g., Mol Ther. 2012 April; 20(4):699-708. doi: 10.1038/mt.2011.287. Epub 2012 Jan. 24. The AAV vector toolkit: poised at the clinical crossroads. Asokan A l, Schaffer D V, Samulski R J.). Methods for producing and using pseudotyped rAAV vectors are known in the art (see, e.g., Duan et al., J. Virol., 75:7662-7671, 2001; Halbert et al., J. Virol., 74:1524-1532, 2000; Zolotukhin et al., Methods, 28:158-167, 2002; and Auricchio et al., Hum. Molec. Genet., 10:3075-3081, 2001).

Methods of making or packaging rAAV particles are known in the art and reagents are commercially available (see, e.g., Zolotukhin et al. Production and purification of serotype 1, 2, and 5 recombinant adeno-associated viral vectors. Methods 28 (2002) 158-167; and U.S. Patent Publication Numbers US20070015238 and US20120322861, which are incorporated herein by reference; and plasmids and kits available from ATCC and Cell Biolabs, Inc.). For example, a plasmid comprising a gene of interest may be combined with one or more helper plasmids, e.g., that contain a rep gene (e.g., encoding Rep78, Rep68, Rep52 and Rep40) and a cap gene (encoding VP1, VP2, and VP3, including a modified VP2 region as described herein), and transfected into a recombinant cells such that the rAAV particle can be packaged and subsequently purified.

In some embodiments, the packaging is performed in a helper cell or producer cell, such as a mammalian cell or an insect cell. Exemplary mammalian cells include, but are not limited to, HEK293 cells, COS cells, HeLa cells, BHK cells, or CHO cells (see, e.g., ATCC® CRL-1573™, ATCC® CRL-1651™, ATCC® CRL-1650™, ATCC® CCL-2, ATCC® CCL-10™, or ATCC® CCL-61™). Exemplary insect cells include, but are not limited to Sf9 cells (see, e.g., ATCC® CRL-1711™). The helper cell may comprises rep and/or cap genes that encode the Rep protein and/or Cap proteins for use in a method described herein. In some embodiments, the packaging is performed in vitro.

In some embodiments, a plasmid containing comprising the gene of interest is combined with one or more helper plasmids, e.g., that contain a rep gene of a first serotype and a cap gene of the same serotype or a different serotype, and transfected into helper cells such that the rAAV particle is packaged.

In some embodiments, the one or more helper plasmids include a first helper plasmid comprising a rep gene and a cap gene, and a second helper plasmid comprising one or more of the following helper genes: E1a gene, E1b gene, E4 gene, E2a gene, and VA gene. For clarity, helper genes are genes that encode helper proteins E1a, E1b, E4, E2a, and VA. In some embodiments, the cap gene is modified such that one or more of the proteins VP1, VP2 and VP3 do not get expressed. In some embodiments, the cap gene is modified such that VP2 does not get expressed. Methods for making such modifications are known in the art (Lux et al. (2005), J Virology, 79: 11776-87) Helper plasmids, and methods of making such plasmids, are known in the art and commercially available (see, e.g., pDF6, pRep, pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, Pa.; Cellbiolabs, San Diego, Calif.; Agilent Technologies, Santa Clara, Ca: and Addgene, Cambridge, Mass.: pxx6; Grimm et al. (1998), Novel Tools for Production and Purification of Recombinant Adeno associated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081: Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188). Plasmids that encode wild-type AAV coding regions for specific serotypes are also know and available. For example pSub201 is a plasmid that comprises the coding regions of the wild-type AAV2 genome (Samulski et al. (1987), J Virology, 6:3096-3101).

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. In some embodiments, the one or more helper plasmids comprise rep genes, cap genes, and optionally one or more of the adenoviral VA, E2A (DBP), and E4 genes under the transcriptional control of their native promoters. In some embodiments, the one or more helper plasmids comprise cap ORFs (and optionally rep ORFs) for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO4-mediated transfection, lipids or polymeric molecules such as Polyethylenimine (PEI) with the helper plasmid(s) and a plasmid containing a nucleic acid vector described herein. The HEK293 cells are then incubated for at least 60 hours to allow for rAAV particle production. Alternatively, the HEK293 cells are transfected via methods described above with AAV-ITR containing one or more genes of interest, a helper plasmid comprising genes encoding Rep and Cap proteins, and co-infected with a helper virus. Helper viruses are viruses that allow the replication of AAV. Examples of helper virus are adenovirus and herpesvirus.

Alternatively, in another example Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known in the art or described herein, e.g., by iodixanol step gradient, CsCl gradient, chromatography, or polyethylene glycol (PEG) precipitation.

### Methods of Administering

Any one of the chimeric RAAV particles or preparations of particles disclosed herein is used to deliver genetic load to a subject. Accordingly, any one of the chimeric RAAV particles or preparations of particles disclosed herein are administered to a subject. In some embodiments, a particular tissue is targeted. In some embodiments, a rAAV particle of a particular serotype is chose to target a particular tissue based on its tropism. Tissue tropism of different rAAV serotypes are known in the art and can be tested using cells of different tissues.

In some embodiments, “administering” or “administration” means providing a material to a subject in a manner that is pharmacologically useful.

In certain circumstances it will be desirable to deliver the rAAV particles in suitably formulated pharmaceutical compositions disclosed herein either subcutaneously, intraocularly, intravitreally, subretinally, parenterally, intravenously (IV), intracerebro-ventricularly, intramuscularly, intrathecally (IT), intracisternally, orally, intraperitoneally, by oral or nasal inhalation, or by direct injection to one or more cells, tissues, or organs by direct injection. In some embodiments, the administration is a route suitable for systemic delivery, such as by intravenous injection. In some embodiments, “administering” or “administration” means providing a material to a subject in a manner that is pharmacologically useful.

In some embodiments, the number of rAAV particles administered to a subject may be on the order ranging from 10⁶ to 10⁴ particles/ml or 10³ to 10¹⁵ particles/ml, or any values therebetween for either range, such as for example, about 10⁶, 10⁷, 10⁸, 10⁹, 10¹⁰, 10¹¹, 10¹², 10¹³, or 10¹⁴ particles/ml. In one embodiment, rAAV particles of higher than 10¹³ particles/ml are be administered. In some embodiments, the number of rAAV particles administered to a subject may be on the order ranging from 10⁶ to 10¹⁴ vector genomes(vgs)/ml or 10³ to 10¹⁵ vgs/ml, or any values therebetween for either range, such as for example, about 10⁶, 10⁷, 10⁸, 10⁹, 10¹⁰, 10¹¹, 10¹², 10³, or 10¹⁴ vgs/ml. In one embodiment, rAAV particles of higher than 10¹³ vgs/ml are be administered. The rAAV particles can be administered as a single dose, or divided into two or more administrations as may be required to achieve therapy of the particular disease or disorder being treated. In some embodiments, 0.0001 ml to 10 mls are delivered to a subject. In some embodiments, the number of rAAV particles administered to a subject may be on the order ranging from 10⁶-10¹ vg/kg, or any values therebetween, such as for example, about 10⁶, 10⁷, 10⁸, 10⁹, 10¹⁰, 10¹¹, 10¹², 10¹³, or 10¹⁴ vgs/mg. In some embodiments, the number of rAAV particles administered to a subject may be on the order ranging from 10¹²-10¹⁴ vgs/kg.

Aspects of the disclosure relate to methods for use with a subject, such as human or non-human primate subjects. Non-limiting examples of non-human primate subjects include macaques (e.g., cynomolgus or rhesus macaques), marmosets, tamarins, spider monkeys, owl monkeys, vervet monkeys, squirrel monkeys, baboons, gorillas, chimpanzees, and orangutans. In some embodiments, the subject is a human subject. Other exemplary subjects include domesticated animals such as dogs and cats; livestock such as horses, cattle, pigs, sheep, goats, and chickens; and other animals such as mice, rats, guinea pigs, and hamsters.

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Biochemical and Biophysical Studies of rAAV of Different Serotypes

Comparative biochemical and biophysical studies with several AAV serotypes (AAV1/6, AAV2, AAV5, AAV8) were carried out. Serotypes were selected to span the spectrum of similarity and differences in sequence and structure.

It was observed that rAAV of different serotypes exhibit a difference in:

(i) pH-dependent capsid stability (FIG. 1B),

(ii) pH-dependent secondary structural transitions proposed to be VP1u unfolding (FIG. 1A), and

(iii) VP1u exposure dynamics (FIG. 1A).

Circular dichroism (CD) showed a loss of secondary structure, mostly in the α-helical region of the spectrum, as pH is dropped, yet the capsids remain in intact. Signal loss could be related to VP1u unfolding in readiness for externalization from the capsid as pH is dropped (Venkatakrishnan et al, 2013, J Virol 87, 4974-4984).

However, the VP1/2 common region bridging the VP1u and the VP3 common region is highly variable in sequence among the AAVs and is intrinsically disordered. This property would provide the unfolded VP1u the tether needed to thread through the 5-fold channel of the capsid.

However, unexpectedly, the pH sensitivity was dramatically different for the four AAV serotypes screened, AAV1, AAV2, AAV5, and AAV8 (FIG. 1). This suggests that the capsid dynamics associated with trafficking in the endosomal pathway differs for the AAVs. For AAV8, unlike the gradual loss in α-helical secondary structure signal seen with AAV1, or the maintenance of signal in AAV2 and AAV5 at low pH, a much more rapid decrease was observed. This observation led to the realization that a faster linearization of α-helical structure, including for the VP1u, as observed for AAV8, would facilitate faster externalization and subsequent trafficking and hence uncoating of this virus for a faster onset of transgene expression.

Particular amino acids in the VP1u of AAV have been implicated in transduction phenotype, e.g., from the comparison of cellular transduction properties for AAV1 and AAV6, where the L129F difference in VP1u affects transduction efficiency (Limberis et al., 2009, Mol Ther 17, 294-301: Li et al., 2009, Mol Ther 17, 2067-2077), and a comparative study of AAV3B and a chimeric AAV variant, LK03, selected in a humanized mouse model, which differed from AAV3B by only one amino in the VP3 common region but had several differences in VP1u contributed by four other serotypes (Lisowski et al., 2014, Nature 506: 382-386). LK03 exhibited 8-fold greater transduction efficiency compared to AAV. However, larger regions of AAV VP1u have not been previously used to alter externalization and enzyme function, post-cellular entry interactions, and subsequently time of onset of transduction and transduction efficiency.

### Example 2: rAAV VP1u Chimeras to Alter Secondary Structure Transitions

Based on the data discussed in Example 1, it was hypothesized that amino acid differences in the VP1u of AAV serotypes affect secondary structure transitions required for externalization and enzyme function, post-cellular entry interactions, and subsequently time of onset of transduction and transduction efficiency. These possibilities were tested using site-directed mutants and chimeras in which amino acids between serotypes are switched. Clinically relevant AAVs, AAV1, AAV2, AAV5, AAV6, AAV8, and AAV9, with different levels of variation in their VP1u sequence served as models. Biophysical observations of VP1u and capsid dynamics, under varying pH and cation conditions, were correlated with transduction (or infection) efficiency. They are also correlated with tissue tropism and transduction differences to benefit efforts aimed at tailoring AAV to specific cells/tissues and improved therapeutic gene expression.

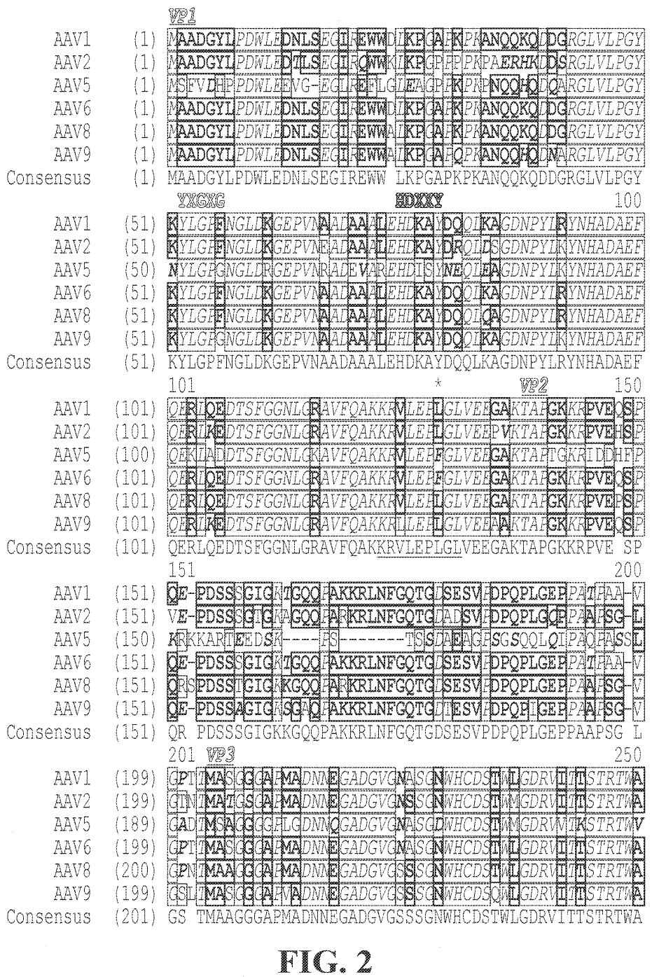

Based on the sequence alignment of the VP1 and VP1/2 amino acids of AAVs of different serotypes (e.g., AAV1, AAV2, AAV5, AAV6, AAV8, and AAV9) (FIG. 2 and FIG. 3), a series of chimeric vectors, in which the VP1u of the AAVs are swapped (Table 1) were generated.

TABLE 1
VP1u Chimeras characterized

| Capsid | Switch |
|---|---|
| AAV2-1VP1u | AAV2 → AAV1 |
| AAV5-1VP1u | AAV5 → AAV1 |
| AAV8-1VP1u | AAV8 → AAV1 |
| AAV9-1VP1u | AAV9 → AAV1 |
| AAV1-8VP1u | AAV1 → AAV8 |
| AAV2-8VP1u | AAV2 → AAV8 |
| AAV5-8VP1u | AAV5 → AAV8 |
| AAV9-8VP1u | AAV9 → AAV8 |

The rAAVs are produced by triple transfection with 3 plasmids and virus-like particles (VLPs) are made using a baculovirus/sf9 expression system. The ability of the baculovirus/sf9 system or transfection to produce protein and assembly capsids were tested, and ability of the rAAVs to infect cells were tested in HEK293 cells. Following this verification, the VLPs and vectors were characterized using experimental approaches including biophysical studies at pH 7.4, 6.0, 5.5, 4.0 &/or heat using Native dot blot assays (w/wo heat: A 1, capsid Mabs), circular dichroism, and differential scanning fluorometry; in vitro studies using cellular fractionation using intact capsids and uncoated genomes; and greed cell assays in different cell lines.

Transduction of cells by chimeric rAAV particles was measured by the expression of the luciferase gene pTR-UF3-Luc 48 hrs post infection at a MOI=105.

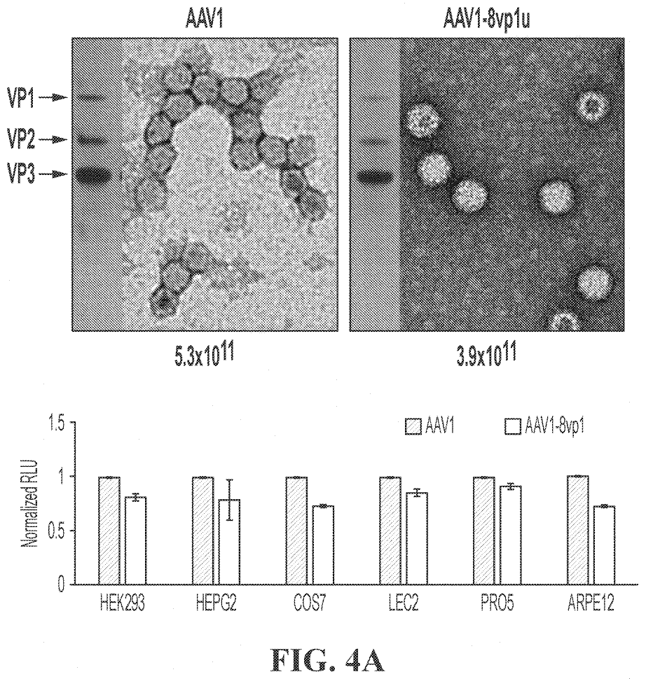

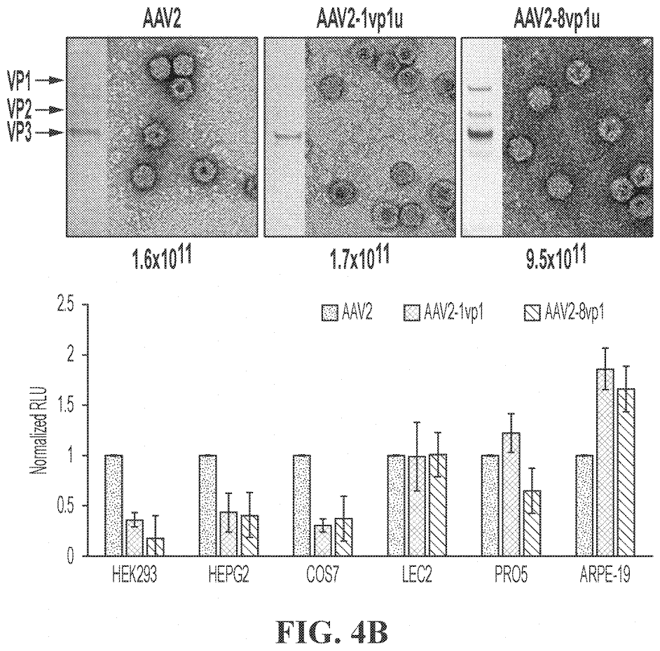

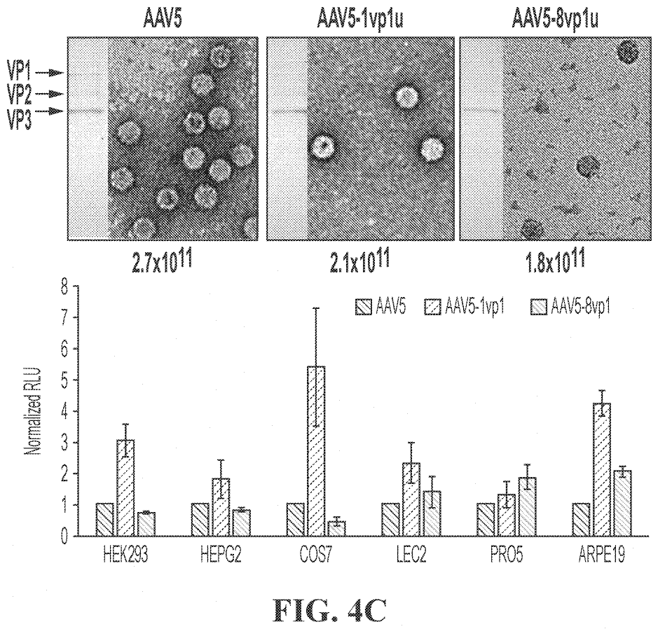

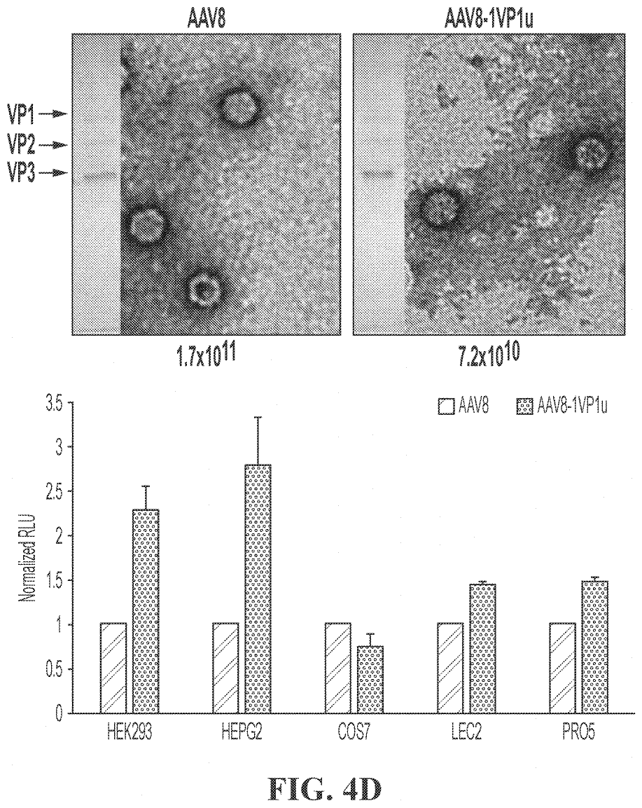

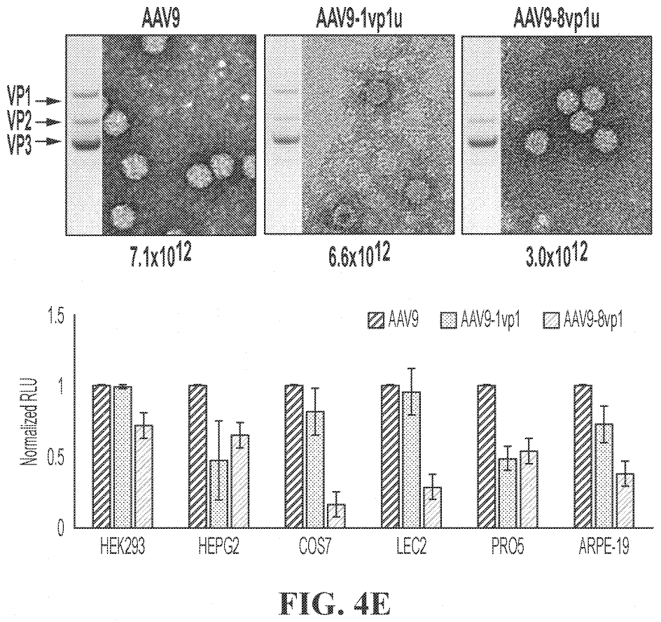

FIGS. 4A-E show how VP1u regions of various serotypes enhance cellular transduction. Improved transduction efficiency in the AAV1-VP1u chimeras did not show improved transduction efficiency, suggesting that the 1VP1u is optimal. FIGS. 4A-E also provide negative stain EMs and silver stained gel showing that capsid particles were properly formed and of appropriate size. The gels show that all three capsid proteins are incorporated into the virus particle in the proper ratio. The three bands represents VP1:VP2:VP3 in the ratio of 1:1:10.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures. Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

1. A chimeric recombinant adeno-associated virus (rAAV) particle comprising a chimeric VP1 capsid protein comprising a backbone of a first serotype and a region of VP1u of a second serotype, wherein the second serotype is AAV serotype 1 (AAV1) or AAV serotype 8 (AAV8) and different from the first serotype, and wherein the chimeric particle has a greater transduction efficiency relative to its wild-type counterpart as measured in a cell.

2. The chimeric rAAV particle of claim 1, wherein the first serotype is 1, 2, 5, 8 or 9.

3. The chimeric rAAV particle of claim 1, wherein the second serotype is AAV1 or AAV8.

4. The chimeric rAAV particle of claim 1, wherein the region of VP1u of the second serotype is at least 5 amino acids long.

5. The chimeric rAAV particle of claim 4, wherein the region of VP1u of the second serotype is at least 50 amino acids long.

6. The chimeric rAAV particle of claim 5, wherein the region of VP1u of the second serotype is at least 100 amino acids long.

7. The chimeric rAAV particle of claim 1, wherein the region of VP1u of the second serotype is amino acids 1-137 of AAV1 or AAV8.

8. The chimeric rAAV particle of claim 7, wherein rAAV particle comprises a region of VP1u of the second serotype that is the VP1u of AAV1 (1VP1u), and wherein the sequence of 1VP1u is SEQ ID NO: 1.

9. The chimeric rAAV particle of claim 7, wherein rAAV particle comprises a region of VP1u of the second serotype that is the VP1u of AAV8 (8VP1u), and wherein the sequence of 8VP1u is SEQ ID NO: 8.

10. The chimeric rAAV particle of claim 1, wherein the region of VP1u of the second serotype comprises a phospholipase A2 domain.

11. The chimeric rAAV particle of claim 1, wherein the region of VP1u of the second serotype comprises residues D24, K84, or both D24 and K84 of AAV1.

12. The chimeric rAAV particle of claim 1, wherein the region of VP1u of the second serotype comprises residues A24, Q84, or both A24 and Q84 of AAV8.

13. The chimeric rAAV particle of claim 1, wherein the chimeric particle has a transduction efficiency that is at least 20% greater relative to its wild-type counterpart.

14. The chimeric rAAV particle of claim 1, wherein the chimeric particle has a transduction efficiency that is greater by at least 4 times relative to its wild-type counterpart.

15. The chimeric rAAV particle of claim 1, wherein the cell is selected from the group consisting of: HEK293, HEPG2, LEC2, PRO5, ARPE19, and COS7.

16. A nucleic acid encoding the VP1 capsid protein of claim 1.

17. A chimeric recombinant adeno-associated virus (rAAV) particle comprising a chimeric VP1 capsid protein comprising:
(i) a backbone of a first serotype, wherein the first serotype is not AAV1; and
(ii) a VP1u of AAV1 (1VP1u) having the amino acid sequence of SEQ ID NO: 1.

18. The chimeric AAV particle of claim 17, wherein the first serotype is 2, 5, 8 or 9.

19. A chimeric recombinant adeno-associated virus (rAAV) particle comprising a chimeric VP1 capsid protein comprising:
(i) a backbone of a first serotype, wherein the first serotype is not AAV8; and
(ii) a VP1u of AAV8 (8VP1u) having the amino acid sequence of SEQ ID NO: 8.

20. The chimeric AAV particle of claim 19, wherein the first serotype is 1, 2, 5, or 9.
